SEQUENCE LISTING (60 sequences)

SEQ ID NO: 1 - protein, 227 aa, Artificial Sequence
  Y349C+T366W
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 227 aa, Artificial Sequence
  D356C+T366S+L368A+Y407V+F405K
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 3 - protein, 227 aa, Artificial Sequence
  Y349C+T366W+F405K
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 4 - protein, 227 aa, Artificial Sequence
  D356C+T366S+L368A+Y407V
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 227 aa, Artificial Sequence
  Y349C+T366W+K409E
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSELTVDKSRWQQGNVFSCSVMHEALHNHYTQKSLSLSPGK

SEQ ID NO: 6 - protein, 227 aa, Artificial Sequence
  Y349C+T366W+K409A
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 7 - protein, 227 aa, Artificial Sequence
  Y349C+T366W+F405K+K360E+Q347E
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPEVCTLPPSRDELTENQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 8 - protein, 227 aa, Artificial Sequence
  D356C+T366S+L368A+Y407V+Q347R
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 227 aa, Artificial Sequence
  Y349C+T366W+F405K+Q347R
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPRVCTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 10 - protein, 227 aa, Artificial Sequence
  D356C+T366S+L368A+Y407V+K360E+Q347E
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSRCELTENQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 227 aa, Artificial Sequence
  Y349C+T366W+K409A+K360E+Q347E
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPEVCTLPPSRDELTENQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 12 - protein, 227 aa, Artificial Sequence
  D356C+T366S+L368A+Y407V+F405K+Q347R
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRCELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 13 - protein, 227 aa, Artificial Sequence
  Y349C+T366W+K409A+Q347R
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPRVCTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 14 - protein, 227 aa, Artificial Sequence
  D356C+T366S+L368A+Y407V+F405K+K360E+Q347E
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSRCELTENQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 15 - protein, 227 aa, Artificial Sequence
  T366W+K409A+K392D
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYDTTPPVLDSDGSFFLYSALTVDKSRWQQGNVFSCSVMHEALHNHYTQKSLSLSPGK

SEQ ID NO: 16 - protein, 227 aa, Artificial Sequence
  T366S+L368A+Y407V+D399S+F405K
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLSSDGSFKLVSKLTVDKSRWQQGNVFSCSVMHEALHNHYTQKSLSLSPGK

SEQ ID NO: 17 - protein, 227 aa, Artificial Sequence
  T366W+K409A
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 18 - protein, 227 aa, Artificial Sequence
  T366S+L368G+Y407A+F405K
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFKLASKLTVDKSRWQQGNVFSCSVMHEALHNHYTQKSLSLSPGK

SEQ ID NO: 19 - protein, 227 aa, Artificial Sequence
  T366W+K409A+Y349D
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 20 - protein, 227 aa, Artificial Sequence
  T366S+L368A+Y407V+F405K+E357A
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDALTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 21 - protein, 227 aa, Artificial Sequence
  T366W+K409A+Y349D+S354D
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPDRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 22 - protein, 227 aa, Artificial Sequence
  T366W+F405K
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 23 - protein, 227 aa, Artificial Sequence
  T366S+L368A+Y407V+K409A
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSALTVDKSRWQQGNVFSCSVMHEALHNHYTQKSLSLSPGK

SEQ ID NO: 24 - protein, 227 aa, Artificial Sequence
  T366W+F405K+D399S
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLSSDGSFKLYSKLTVDKSRWQQGNVFSCSVMHEALHNHYTQKSLSLSPGK

SEQ ID NO: 25 - protein, 227 aa, Artificial Sequence
  T366S+L368A+Y407V+K409A+K392D
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYDTTPPVLDSDGSFFLVSALTVDKSRWQQGNVFSCSVMHEALHNHYTQKSLSLSPGK

SEQ ID NO: 26 - protein, 227 aa, Artificial Sequence
  T366S+L368G+Y407A+K409A
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFFLASALTVDKSRWQQGNVFSCSVMHEALHNHYTQKSLSLSPGK

SEQ ID NO: 27 - protein, 227 aa, Artificial Sequence
  T366W +F405K +Y349D
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 28 - protein, 227 aa, Artificial Sequence
  T366S+L368A+Y407V +K409A +E357A
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDALTKNQVSLSCAVKGFYPSDIAVEWESNGQPENNYKTTPPVLDSDGSFFLVSALTVDKSRWQQGNVFSCSVMHEALHNHYTQKSLSLSPGK

SEQ ID NO: 29 - protein, 227 aa, Artificial Sequence
  T366W+F405K+Y349D+S354D
DKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPDRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 30 - protein, 227 aa, Artificial Sequence
  wildtype human IgG1-Fc region amino acid
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 681 bp, Artificial Sequence
  Fc gene fragment
gacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgagaaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 32 - DNA, 741 bp, Artificial Sequence
  polynucleotide fragment encoding a mouse kappaIII signal peptide
atggagaccgacaccctgctgctgtgggtgctgctgctgtgggtgcccggcagcaccggcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgagaaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 33 - DNA, 1473 bp, Artificial Sequence
  gene  of ScFv-Fc fusion protein
atggagaccgacaccctgctgctgtgggtgctgctgctgtgggtgcccggcagcaccggcgaggtgcagctgctggagagcggcggcggcgtggtgcagcccggccgcagcctgcgcctgagctgcatcgccagcggcttcaccttcagcagctaccccatgacctgggtgcgccaggcccccggcaagggcctggagtgggtggccagcatcagctacgacggcagctacaagtacaaggccgacagcatgaagggccgcctgaccatcagccgcgacaacagcaagaacaccctgtacctggagatgaacagcctgaccgccgaggacaccgccgtgtactactgcgcccgcaccgccttcttcaacgcctacgacttctggggccagggcaccctggtgaccgtgagcagcgccagcaccaagggccccagcgtgggcggcggcggcagcggcggcggcggcagcgagatcgtgatgacccagagccccgccaccctgagcgtgagccccggcgagcgcgccaccctgagctgccgcgccagccagagcgtgcgcagcaacctggcctggtaccagcagaagcccggccaggccccccgcctgctgatctacgccgccagcacccgcgccaccggcatccccgcccgcttcagcggcagcggcagcggcaccgagttcaccctgaccatcagcagcctgcagagcgaggacttcgccgtgtactactgccagcagtacaacgagtggttccgcaccagcggccagggcaccaaggtggagatcaagcgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgagaaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 34 - protein, 471 aa, Artificial Sequence
  ScFv-Fc fusion protein
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1121 bp, Artificial Sequence
  a fusion gene fragment encoding the fusion protein VhH-Fc
atggagaccgacaccctgctgctgtgggtgctgctgctgtgggtgcccggcagcaccggccaggtgcagctgcaggagtctgggggaggctcggtgcaggctggagggtctctgagactctcctgtgcagcctctgaatacatctacagtagctactgcatggcctggttccgccaggctccagggaaggagcgcgagggggtcgcagttattgggagtgatggtagcacaagctacgcagactccgtgaaaggccgattcaccatctccaaagacaacgccaagaacactctgtatctgcaaatgaacagcctgaaacctgaggacactgccatgtactactgtgcggccatcggtggttactgctaccaaccaccctatgagtaccagtactggggccaggggacccaggtcaccgtctcccagaaccgaaaagcagcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgagaaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 36 - protein, 354 aa, Artificial Sequence
  fusion protein VhH-Fc
QVQLQESGGGSVQAGGSLRLSCAASEYIYSSYCMAWFRQAPGKEREGVAVIGSDGSTSYADSVKGRFTISKDNAKNTLYLQMNSLKPEDTAMYYCAAIGGYCYQPPYEYQYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, Artificial Sequence
  amino acid sequence of Erb-LC
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 642 bp, Artificial Sequence
  gene of Erb-LC
gacatcctgctgacccagagccccgtgatcctgagcgtgagccccggcgagcgcgtgagcttcagctgccgcgccagccagagcatcggcaccaacatccactggtaccagcagcgcaccaacggcagcccccgcctgctgatcaagtacgccagcgagagcatcagcggcatccccagccgcttcagcggcagcggcagcggcaccgacttcaccctgagcatcaacagcgtggagagcgaggacatcgccgactactactgccagcagaacaacaactggcccaccaccttcggcgccggcaccaagctggagctgaagcgcaccgtggccgcccccagcgtgttcatcttcccccccagcgacgagcagctgaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 39 - protein, 449 aa, Artificial Sequence
  amino acid sequences of Erb-Fc9
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 40 - DNA, 1347 bp, Artificial Sequence
  gene of Erb-Fc9
caggtgcagctgaagcagagcggccccggcctggtgcagcccagccagagcctgagcatcacctgcaccgtgagcggcttcagcctgaccaactacggcgtgcactgggtgcgccagagccccggcaagggcctggagtggctgggcgtgatctggagcggcggcaacaccgactacaacacccccttcaccagccgcctgagcatcaacaaggacaacagcaagagccaggtgttcttcaagatgaacagcctgcagagcaacgacaccgccatctactactgcgcccgcgccctgacctactacgactacgagttcgcctactggggccagggcaccctggtgaccgtgagcgccgccagcactaaggggccctctgtgtttccactcgccccttctagcaaaagcacttccggaggaactgccgctctgggctgtctggtgaaagattacttccccgaaccagtcactgtgtcatggaactctggagcactgacatctggagttcacacctttcctgctgtgctgcagagttctggactgtactccctgtcatctgtggtcaccgtgccatcttcatctctggggacccagacctacatctgtaacgtgaaccacaaaccctccaacacaaaagtggacaaacgagtcgaaccaaaatcttgtgacaaaacccacacatgcccaccgtgcccagctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagtcgggatgagctgaccaagaaccaggtcagcctgtggtgcctggtcaaaggcttctatcccagcgacatcgccgtggagtgggagagcaatgggcagccggagaacaactacaagaccacgcctcccgtgttggactccgacggctccttcttcctctacagcgcgctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - protein, 15 aa, Artificial Sequence
  linker 1
GGGGSGGGGSGGGGS

SEQ ID NO: 42 - protein, 582 aa, Artificial Sequence
  amino acid sequence of (IL10)2-Fc6
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFKLASKLTVDKSRWQQGNVFSCSVMHEALHNHYTQKSLSLSPGK

SEQ ID NO: 43 - DNA, 1746 bp, Artificial Sequence
  gene of (IL10)2-Fc6
agccccggccagggcacacagtccgagaacagctgcacccactttcccggcaacctgcctaacatgctgagggacctgagggacgccttcagcagggtgaagaccttcttccagatgaaggaccagctggataacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgaggctgaggctgaggaggtgccacaggttcctgccctgtgagaacaaatccaaggccgtggagcaggtgaagaacgccttcaacaagctgcaggaaaagggcatctacaaggccatgagcgagttcgacatctttatcaactatatcgaggcctacatgacaatgaagatcaggaacggcggcggcggcagcgggggcggcggcagcggaggaggcggcagcagccccggccagggcacacagtccgagaacagctgcacccactttcccggcaacctgcctaacatgctgagggacctgagggacgccttcagcagggtgaagaccttcttccagatgaaggaccagctggataacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgaggctgaggctgaggaggtgccacaggttcctgccctgtgagaacaaatccaaggccgtggagcaggtgaagaacgccttcaacaagctgcaggaaaagggcatctacaaggccatgagcgagttcgacatctttatcaactatatcgaggcctacatgacaatgaagatcaggaacggcggcggcggcagcgggggcggcggcagcggaggaggcggcagcgagcctaagtccagcgacaagacccacacctgccccccttgccccgctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatcccgggatgagctgaccaagaaccaggtcagcctgagttgcggggtcaaaggcttctatcccagcgacatcgccgtggagtgggagagcaatgggcagccggagaacaactacaagaccacgcctcccgtgttggactccgacggctccttcaagctcgccagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 407 aa, Artificial Sequence
  amino acid sequence of IL10-Fc
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1221 bp, Artificial Sequence
  gene of IL10-Fc
agccccggccagggcacacagtccgagaacagctgcacccactttcccggcaacctgcctaacatgctgagggacctgagggacgccttcagcagggtgaagaccttcttccagatgaaggaccagctggataacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgaggctgaggctgaggaggtgccacaggttcctgccctgtgagaacaaatccaaggccgtggagcaggtgaagaacgccttcaacaagctgcaggaaaagggcatctacaaggccatgagcgagttcgacatctttatcaactatatcgaggcctacatgacaatgaagatcaggaacggcggcggcggcagcgggggcggcggcagcggaggaggcggcagcgagcctaagtccagcgacaagacccacacctgccccccttgccccgctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 46 - DNA, 681 bp, Artificial Sequence
  gene of Fc9
gacaaaactcacacatgcccaccgtgcccagctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagtcgggatgagctgaccaagaaccaggtcagcctgtggtgcctggtcaaaggcttctatcccagcgacatcgccgtggagtgggagagcaatgggcagccggagaacaactacaagaccacgcctcccgtgttggactccgacggctccttcttcctctacagcgcgctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - protein, 8 aa, Artificial Sequence
  Kb-binding peptide antigen
SIYRYYGL

SEQ ID NO: 48 - protein, 11 aa, Artificial Sequence
  Cetuximab light chain CDR1
RASQSIGTNIH

SEQ ID NO: 49 - protein, 7 aa, Artificial Sequence
  Cetuximab light chain CDR2
YASESIS

SEQ ID NO: 50 - protein, 9 aa, Artificial Sequence
  Cetuximab light chain CDR3
QQNNNWPTT

SEQ ID NO: 51 - protein, 107 aa, Artificial Sequence
  Cetuximab light chain variable region
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 52 - protein, 5 aa, Artificial Sequence
  Cetuximab heavy chain CDR1
NYGVH

SEQ ID NO: 53 - protein, 16 aa, Artificial Sequence
  Cetuximab heavy chain CDR2
VIWSGGNTDYNTPFTS

SEQ ID NO: 54 - protein, 11 aa, Artificial Sequence
  Cetuximab heavy chain CDR3
ALTYYDYEFAY

SEQ ID NO: 55 - protein, 119 aa, Artificial Sequence
  Cetuximab heavy chain variable region
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 56 - protein, 160 aa, Artificial Sequence
  IL10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 57 - DNA, 480 bp, Artificial Sequence
  gene of IL10
agccccggccagggcacacagtccgagaacagctgcacccactttcccggcaacctgcctaacatgctgagggacctgagggacgccttcagcagggtgaagaccttcttccagatgaaggaccagctggataacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgaggctgaggctgaggaggtgccacaggttcctgccctgtgagaacaaatccaaggccgtggagcaggtgaagaacgccttcaacaagctgcaggaaaagggcatctacaaggccatgagcgagttcgacatctttatcaactatatcgaggcctacatgacaatgaagatcaggaac

SEQ ID NO: 58 - protein, 335 aa, Artificial Sequence
  (IL10)2
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 59 - DNA, 1005 bp, Artificial Sequence
  gene of (IL10)2
agccccggccagggcacacagtccgagaacagctgcacccactttcccggcaacctgcctaacatgctgagggacctgagggacgccttcagcagggtgaagaccttcttccagatgaaggaccagctggataacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgaggctgaggctgaggaggtgccacaggttcctgccctgtgagaacaaatccaaggccgtggagcaggtgaagaacgccttcaacaagctgcaggaaaagggcatctacaaggccatgagcgagttcgacatctttatcaactatatcgaggcctacatgacaatgaagatcaggaacggcggcggcggcagcgggggcggcggcagcggaggaggcggcagcagccccggccagggcacacagtccgagaacagctgcacccactttcccggcaacctgcctaacatgctgagggacctgagggacgccttcagcagggtgaagaccttcttccagatgaaggaccagctggataacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgaggctgaggctgaggaggtgccacaggttcctgccctgtgagaacaaatccaaggccgtggagcaggtgaagaacgccttcaacaagctgcaggaaaagggcatctacaaggccatgagcgagttcgacatctttatcaactatatcgaggcctacatgacaatgaagatcaggaac

SEQ ID NO: 60 - protein, 5 aa, Artificial Sequence
  hinge
EPKSS

(19) United States
(12) Patent Application Publication
(10) Pub. No.: US 2023/0241236 A1
(43) Pub. Date: Aug. 3, 2023

(54) METHODS AND COMPOSITIONS FOR CANCER TREATMENT

(71) Applicant: DINGFU BIOTARGET, CO., LTD., Suzhou (CN)

(72) Inventors: Ting XU, Suzhou (CN); Kai FU, Suzhou (CN); Jianjian PENG, Suzhou (CN); Shilong FU, Suzhou (CN); Jian DING, Suzhou (CN); Liyao ZHOU, Suzhou (CN)

(73) Assignee: DINGFU BIOTARGET, CO., LTD., Suzhou (CN)

(21) Appl. No.: 18/003,484

(22) PCT Filed: Jun. 28, 2021
(86) PCT No.: PCT/CN2021/102732

§ 371 Date: Dec. 27, 2022

(30) Foreign Application Priority Data
Jun. 28, 2020 (WO) PCT/CN2020/098580

(51) Int. Cl. A61K 47/68; C07K 14/54; C07K 16/28; A61K 31/513; A61K 31/555; A61K 45/06; A61P 35/00

(52) U.S. Cl. CPC A61K 47/6813; C07K 14/5428; C07K 16/2863; A61K 31/513; A61K 31/555; A61K 47/6849; A61K 45/06; A61P 35/00

(57) ABSTRACT

A composition including an immunoconjugate and a chemotherapeutic agent. The immunoconjugate includes 1) one or more interleukins, and 2) an Fc domain consisting of a first Fc subunit and a second Fc subunit. The first Fc subunit associates with the second Fc subunit to form a dimer. The one or more interleukins are fused to the Fc domain and the chemotherapeutic agent includes a fluorouracil and/or an oxaliplatin.

### BACKGROUND

Although immune responses against tumor antigens can be detected (Disis et al. (1997) J. Clin. Oncol. 15: 3363-3367), malignant cells causing diseases often fail to elicit an immune response that leads to rejection. Studies have demonstrated that it is possible to enhance the immunogenicity of tumor cells by introducing immunoregulatory molecules such as cytokines and costimulatory molecules into them; however, eradication of residual cancer cells may require the targeting of widely scattered micrometastatic tumor deposits that are not accessible to direct gene transfer. In addition, the expression and stability of the immunoregulatory molecules introduced are often far from satisfactory. Immunoregulators, such as cytokines, produced by cells of the immune system can, directly or indirectly, activate the cells of the adaptive immune response and can play an important role in eliciting protective antitumor immunity. The innate immune system can be triggered by bacterial products or “danger” signals that lead to the release of proinflammatory cytokines, such as interleukins.

Multiple studies have shown that immunoregulators may be useful in exerting antitumor effects in both animal models and cancer patients. However, short half-life and systemic toxicity related with application of the immunoregulators have greatly limited their usage.

Besides immunotherapy, surgery, chemotherapy, hormonal therapy and radiation therapy have also been used to treat cancer (see, for example, Stockdale, 1998, “Principles of Cancer Patient Management”, in Scientific American: Medicine, vol. 3, Rubenstein and Federman, eds., Chapter 12, Section IV). However, surgery may be impossible or unacceptable due to patient health conditions or advanced disease stages, and frequently, cancer cells cannot be completely removed from the patients after surgery. Hormonal therapy is rarely given as a single agent and although can be effective, is often used to prevent or delay recurrence of cancer after other treatments have removed the majority of the cancer cells. Further, patients may develop drug resistance pretty quickly to chemotherapy agents.

Thus, novel and effective therapies for cancer treatment are still much desired.

### SUMMARY OF THE INVENTION

The present disclosure provides compositions and methods for treating cancer, comprising the use of an immunoconjugate in combination with a chemotherapeutic agent, the inventions of the present disclosure showed significant synergistic effects in cancer treatment.

In one aspect, the present disclosure provides a composition comprising an immunoconjugate and a chemotherapeutic agent, wherein: said immunoconjugate comprises 1) one or more interleukins, and 2) an Fc domain consisting of a first Fc subunit and a second Fc subunit, said first Fc subunit associates with said second Fc subunit to form a dimer; said one or more interleukins are fused to said Fc domain; and wherein said chemotherapeutic agent comprises a fluorouracil and/or an oxaliplatin.

In some embodiments, at least one of said one or more interleukins is fused to an amino-terminal amino acid of said Fc domain.

In some embodiments, said immunoconjugate comprises two or more interleukins.

In some embodiments, at least two of said two or more interleukins are fused to an amino-terminal amino acid of said Fc domain.

In some embodiments, one or more of said interleukins is fused to said Fc domain through a peptide linker and/or an immunoglobulin hinge region.

In some embodiments, at least two of said two or more interleukins are fused to each other through a peptide linker to form an interleukin dimer.

In some embodiments, at least one said interleukin dimer is fused to an amino-terminal amino acid of said Fc domain. For example, at least one said interleukin dimer may be fused to an amino-terminal amino acid of said Fc domain through a peptide linker or an immunoglobulin hinge region.

In some embodiments, said two or more interleukins are two or more copies of the same interleukin.

In some embodiments, said two or more interleukins are two or more copies of IL10.

In some embodiments, said one or more interleukins comprise one or more IL10.

In some embodiments, said immunoconjugate further comprises a targeting moiety fused to said Fc domain, wherein said targeting moiety exhibits binding specificity to a tumor antigen

In some embodiments, said targeting moiety is fused to an amino-terminal amino acid of said Fc domain.

In some embodiments, said targeting moiety is fused to said Fc domain through a peptide linker or an immunoglobulin hinge region.

In some embodiments, said targeting moiety comprises an antigen binding domain of an antibody

In some embodiments, said antigen binding domain of an antibody is a Fab moiety.

In some embodiments, said tumor antigen is EGFR.

In some embodiments, said targeting moiety comprises an antigen-binding domain of an anti-EGFR antibody.

In some embodiments, said anti-EGFR antibody is cetuximab.

In some embodiments, targeting moiety comprises the heavy chain CDR1-3 of cetuximab, HCDR1 comprises an amino acid sequence as set forth in SEQ ID NO: 52, HCDR2 of cetuximab comprises an amino acid sequence as set forth in SEQ ID NO: 53, and HCDR3 comprises an amino acid sequence as set forth in SEQ ID NO: 54.

In some embodiments, said targeting moiety comprises the light chain CDR1-3 of cetuximab, LCDR1 comprises an amino acid sequence as set forth in SEQ ID NO: 48, LCDR2 comprises an amino acid sequence as set forth in SEQ ID NO: 49, and LCDR3 comprises an amino acid sequence as set forth in SEQ ID NO: 50.

In some embodiments, said targeting moiety comprises the heavy chain variable region of cetuximab, and the heavy chain variable region comprises an amino acid sequence as set forth in SEQ ID NO: 55.

In some embodiments, said targeting moiety comprises the light chain variable region of cetuximab, and the light chain variable region comprises an amino acid sequence as set forth in SEQ ID NO: 51.

In some embodiments, said Fc domain is an IgG Fc domain.

In some embodiments, said IgG is an IgG1.

In some embodiments, said IgG is a human IgG1

In some embodiments, said immunoconjugate is an asymmetric immunoconjugate comprising a first member and a second member different from said first member, wherein said first member comprises said first Fc subunit, and said second member comprises said one or more interleukins fused to said second Fc subunit, and said first Fc subunit associates with said second Fc subunit to form said dimer of the Fc domain.

In some embodiments, in said second member, at least one of said one or more interleukins is fused to the amino-terminal amino acid of said second Fc subunit.

In some embodiments, in said second member, at least two of said one or more interleukins are fused to each other to form an interleukin dimer, and said interleukin dimer is further fused to the amino-terminal amino acid of said second Fc subunit.

In some embodiments, said first member further comprises said targeting moiety fused to said first Fc subunit.

In some embodiments, in said first member, said targeting moiety is fused to the amino-terminal amino acid of said first Fc subunit

In some embodiments, said first Fc subunit is different from said second Fc subunit, and said Fc domain comprises a modification promoting heterodimerization between said first Fc subunit and said second Fc subunit.

In some embodiments, said first Fc subunit comprises a first modification, and said second Fc subunit comprises a second modification

In some embodiments, said first modification comprises an amino acid substitution at position T366, and an amino acid substitution at one or more positions selected from the group consisting of: Y349, F405, K409, D399, K360, Q347, K392 and 5354, wherein the position of the amino acid is determined according to the EU index of the KABAT number.

In some embodiments, the amino acid substitution comprised by the first modification is selected from the group consisting of: Y349C, Y349D, D399S, F405K, K360E, K409A, K409E, Q347E, Q347R, S354D, K392D and T366W, wherein the position of the amino acid is determined according to the EU index of the KABAT number.

In some embodiments, said first modification comprises 2-5 amino acid substitutions.

In some embodiments, said first modification comprises an amino acid substitution at a group of positions selected from any of the following groups: 1) Y349 and T366; 2) Y349, T366 and F405; 3) Y349, T366 and K409; 4) Y349, T366, F405, K360 and Q347; 5) Y349, T366, F405 and Q347; 6) Y349, T366, K409, K360 and Q347; 7) Y349, T366, K409 and Q347; 8) T366, K409 and K392; 9) T366 and K409; 10) T366, K409, Y349 and 5354; 11) T366 and F405; 12) T366, F405 and D399; and 13) T366, F405, Y349 and S354; wherein the position of the amino acid is determined according to the EU index of the KABAT number.

In some embodiments, said first modification comprises a group of amino acid substitutions selected from any of the following groups: 1) Y349C and T366W; 2) Y349C, T366W and F405K; 3) Y349C, T366W and K409E; 4) Y349C, T366W and K409A; 5) Y349C, T366W, F405K, K360E and Q347E; 6) Y349C, T366W, F405K and Q347R; 7) Y349C, T366W, K409A, K360E and Q347E; 8) Y349C, T366W, K409A and Q347R; 9) T366W, K409A and K392D; 10) T366W and K409A; 11) T366W, K409A and Y349D; 12) T366W, K409A, Y349D and S354D; 13) T366W and F405K; 14) T366W, F405K and D399S; 15) T366W, F405K and Y349D; and 16) T366W, F405K, Y349D and S354D; wherein the position of the amino acid is determined according to the EU index of the KABAT number.

In some embodiments, said second modification comprises amino acid substitutions at positions T366, L368 and Y407, as well as an amino acid substitution at one or more positions selected from the group consisting of D356, D399, E357, F405, K360, K392, K409 and Q347, wherein the position of the amino acid is determined according to the EU index of the KABAT number.

In some embodiments, the amino acid substitution comprised by the second modification is selected from the group consisting of D356C, D399S, E357A, F405K, K360E, K392D, K409A, L368A, L368G, Q347E, Q347R, T366S, Y407A and Y407V, wherein the position of the amino acid is determined according to the EU index of the KABAT number.

In some embodiments, the second modification comprises 4-6 amino acid substitutions.

In some embodiments, the second modification comprises an amino acid substitution at a group of positions selected from any of the following groups: 1) D356, T366, L368, Y407 and F405; 2) D356, T366, L368 and Y407; 3) D356, T366, L368, Y407 and Q347; 4) D356, T366, L368, Y407, K360 and Q347; 5) D356, T366, L368, Y407, F405 and Q347; 6) D356, T366, L368, Y407, F405, K360 and Q347; 7) T366, L368, Y407, D399 and F405; 8) T366, L368, Y407 and F405; 9) T366, L368, Y407, F405 and E357; 10) T366, L368, Y407 and K409; 11) T366, L368, Y407, K409 and K392; and 12) T366, L368, Y407, K409 and E357; wherein the position of the amino acid is determined according to the EU index of the KABAT number.

In some embodiments, the second modification comprises a group of amino acid substitutions selected from any of the following groups: 1) D356C, T366S, L368A, Y407V and F405K; 2) D356C, T366S, L368A and Y407V; 3) D356C, T366S, L368A, Y407V and Q347R; 4) D356C, T366S, L368A, Y407V, K360E and Q347E; 5) D356C, T366S, L368A, Y407V, F405K and Q347R; 6) D356C, T366S, L368A, Y407V, F405K, K360E and Q347E; 7) T366S, L368A, Y407V, D399S and F405K; 8) T366S, L368G, Y407A and F405K; 9) T366S, L368A, Y407V, F405K and E357A; 10) T366S, L368A, Y407V and K409A; 11) T366S, L368A, Y407V, K409A and K392D; 12) T366S, L368G, Y407A and K409A; 13) T366S, L368A, Y407V, K409A and E357A; wherein the position of the amino acid is determined according to the EU index of the KABAT number.

In some embodiments, said first Fc subunit comprises said first modification, said second Fc subunit comprises said second modification, and said first modification and said second modification comprise an amino acid substitution at a group of positions selected from any of said following groups: 1) said first modification: Y349 and T366; and said second modification: D356, T366, L368, Y407 and F405; 2) said first modification: Y349, T366 and F405; and said second modification: D356, T366, L368 and Y407; 3) said first modification: Y349, T366 and K409; and said second modification: D356, T366, L368, Y407 and F405; 4) said first modification: Y349, T366, F405, K360 and Q347; and said second modification: D356, T366, L368, Y407 and Q347; 5) said first modification: Y349, T366, F405 and Q347; and said second modification: D356, T366, L368, Y407, K360 and Q347; 6) said first modification: Y349, T366, K409, K360 and Q347; and said second modification: D356, T366, L368, Y407, F405 and Q347; 7) said first modification: Y349, T366, K409 and Q347; and said second modification: D356, T366, L368, Y407, F405, K360 and Q347; 8) said first modification: T366, K409 and K392; and said second modification: T366, L368, Y407, D399 and F405; 9) said first modification: T366 and K409; and said second modification: T366, L368, Y407 and F405; 10) said first modification: T366, K409 and Y349; and said second modification: T366, L368, Y407, F405 and E357; 11) said first modification: T366, K409, Y349 and 5354; and said second modification: T366, L368, Y407, F405 and E357; 12) said first modification: T366 and F405; and said second modification: T366, L368, Y407 and K409; 13) said first modification: T366, F405 and D399; and said second modification: T366, L368, Y407, K409 and K392; 14) said first modification: T366, F405 and Y349; and said second modification: T366, L368, Y407, K409 and E357; 15) said first modification: T366, F405, Y349 and S354; and said second modification: T366, L368, Y407, K409 and E357; wherein the position of the amino acid is determined according to the EU index of the KABAT number.

In some embodiments, said first Fc subunit comprises said first modification, said second Fc subunit comprises said second modification, wherein said first modification and said second modification comprise a group of amino acid substitutions selected from any of said following groups: 1) said first modification: Y349C and T366W; and said second modification: D356C, T366S, L368A, Y407V and F405K; 2) said first modification: Y349C, T366W and F405K; and said second modification: D356C, T366S, L368A and Y407V; 3) said first modification: Y349C, T366W and K409E; and said second modification: D356C, T366S, L368A, Y407V and F405K; 4) said first modification: Y349C, T366W and K409A; and said second modification: D356C, T366S, L368A, Y407V and F405K; 5) said first modification: Y349C, T366W, F405K, K360E and Q347E; and said second modification: D356C, T366S, L368A, Y407V and Q347R; 6) said first modification: Y349C, T366W, F405K and Q347R; and said second modification: D356C, T366S, L368A, Y407V, K360E and Q347E; 7) said first modification: Y349C, T366W, K409A, K360E and Q347E; and said second modification: D356C, T366S, L368A, Y407V, F405K and Q347R; 8) said first modification: Y349C, T366W, K409A and Q347R; and said second modification: D356C, T366S, L368A, Y407V, F405K, K360E and Q347E; 9) said first modification: T366W, K409A and K392D; and said second modification: T366S, L368A, Y407V, D399S and F405K; 10) said first modification: T366W and K409A; and said second modification: T366S, L368G, Y407A and F405K; 11) said first modification: T366W, K409A and Y349D; and said second modification: T366S, L368A, Y407V, F405K and E357A; 12) said first modification: T366W, K409A, Y349D and S354D; and said second modification: T366S, L368A, Y407V, F405K and E357A; 13) said first modification: T366W and F405K; and said second modification: T366S, L368A, Y407V and K409A; 14) said first modification: T366W, F405K and D399S; and said second modification: T366S, L368A, Y407V, K409A and K392D; 15) said first modification: T366W and F405K; and said second modification: T366S, L368G, Y407A and K409A; 16) said first modification: T366W, F405K and Y349D; and said second modification: T366S, L368A, Y407V, K409A and E357A; 17) said first modification: T366W, F405K, Y349D and S354D; and said second modification: T366S, L368A, Y407V, K409A and E357A; wherein the position of the amino acid is determined according to the EU index of the KABAT number.

In some embodiments, said first Fc subunit comprises said first modification, said second Fc subunit comprises said second modification, said first modification comprises said amino acid substitutions T366W and K409A, and said second modification comprises said amino acid substitutions T366S, L368G, Y407A and F405K, wherein the position of the amino acid is determined according to the EU index of the KABAT number.

In some embodiments, wherein said first member does not comprise any interleukin.

In some embodiments, said first Fc subunit comprises an amino acid sequence as set forth in any one of SEQ ID NO: 17.

In some embodiments, said second Fc subunit comprises an amino acid sequence as set forth in any one of SEQ ID NO: 18

In some embodiments, said interleukin is a human interleukin.

In some embodiments, said interleukin comprises an amino acid sequence as set forth in any one of SEQ ID NO: 56.

In some embodiments, said immunoconjugate comprises a first polypeptide chain, a second polypeptide chain and a third polypeptide chain, said first polypeptide chain comprises an amino acid sequence as set forth in SEQ ID NO: 37, said second polypeptide chain comprises an amino acid sequence as set forth in SEQ ID NO: 39, and said third polypeptide chain comprises an amino acid sequence as set forth in SEQ ID NO:42.

In some embodiments, said first member comprises a first polypeptide chain and a second polypeptide chain, said second member comprises a third polypeptide chain, said first polypeptide chain comprises an amino acid sequence as set forth in SEQ ID NO: 37, said second polypeptide chain comprises an amino acid sequence as set forth in SEQ ID NO: 39, and said third polypeptide chain comprises an amino acid sequence as set forth in SEQ ID NO: 42.

In some embodiments, said first member comprises a first polypeptide chain, and said second member comprises a second polypeptide chain, said first polypeptide chain comprises an amino acid sequence as set forth in SEQ ID NO: 17, and said second polypeptide chain comprises an amino acid sequence as set forth in SEQ ID NO: 42.

In some embodiments, said fluorouracil comprises 5-Fu.

In some embodiments, said chemotherapeutic agent further comprises a folinic acid.

In some embodiments, said chemotherapeutic agent comprises a tetrahydrofolate and/or a calcium leucovorin.

In some embodiments, said chemotherapeutic agent comprises a FOLFOX regimen.

In some embodiments, said fluorouracil and said oxaliplatin are not mixed with each other in the composition

In other aspects, the present disclosure provides an immunoconjugate for the use of treating cancer in combination with a chemotherapeutic agent, wherein said immunoconjugate is as defined in the present disclosure, and said chemotherapeutic agent is as defined in the present disclosure.

In some embodiments, said cancer is selected from pancreatic cancer and colorectal cancer.

In some embodiments, said pancreatic cancer is metastatic pancreatic cancer.

In some embodiments, said colorectal cancer is metastatic colorectal cancer.

In some embodiments, said cancer or a cell thereof has elevated expression of EGFR

In other aspects, the present disclosure provides use of an immunoconjugate in combination with a chemotherapeutic agent in the preparation of a medicament for treating cancer in a subject in need thereof, wherein said immunoconjugate is as defined in the present disclosure, and said chemotherapeutic agent is defined in the present disclosure

In some embodiments, said cancer is selected from pancreatic cancer and colorectal cancer.

In some embodiments, said pancreatic cancer is metastatic pancreatic cancer.

In some embodiments, said colorectal cancer is metastatic colorectal cancer.

In some embodiments, said cancer or a cell thereof has elevated expression of EGFR.

In other aspects, the present disclosure provides a method for treating cancer in a subject in need thereof, comprising administering to said subject (a) an effective amount of an immunoconjugate as defined in the present disclosure, and (b) an effective amount of a chemotherapeutic agent as defined in the present disclosure.

In some embodiments, said immunoconjugate is administered to said subject subsequent to administration of said chemotherapeutic agent.

In some embodiments, said immunoconjugate is administered to said subject no more than 10 days after administration of said chemotherapeutic agent.

In some embodiments, said immunoconjugate is administered to said subject no more than 3 days after administration of said chemotherapeutic agent.

In some embodiments, said immunoconjugate is administered to said subject for two or more times.

In some embodiments, said cancer is selected from pancreatic cancer, melanoma and colorectal cancer.

In some embodiments, said pancreatic cancer is metastatic pancreatic cancer.

In some embodiments, said colorectal cancer is metastatic colorectal cancer.

In some embodiments, said cancer is melanoma.

In some embodiments, said cancer or a cell thereof has elevated expression of EGFR.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIGS. 1A-1E illustrate the purification result of the immunoconjugate of the present disclosure, as shown by SDS-PAGE analysis and SEC-HPLC analysis.

FIGS. 2A-2D illustrate the effect of various chemotherapies in combination with the immunoconjugate of the present disclosure.

FIGS. 3A-3C illustrate examples of the immunoconjugate according to the present disclosure.

FIGS. 4A-4B illustrate that the tumor growth was inhibited by Erb-(IL10)₂, FOLFOX and the combination of Erb-(IL10)₂ and FOLFOX.

FIG. 4C illustrates that the survival rate of mice was prolonged by Erb-(IL10)₂, FOLFOX and the combination of Erb-(IL10)₂ and FOLFOX.

FIGS. 5A-5B illustrate that the tumor growth was inhibited by Erb-(IL10)₂, FOLFOX and the combination of Erb-(IL10)₂ and FOLFOX.

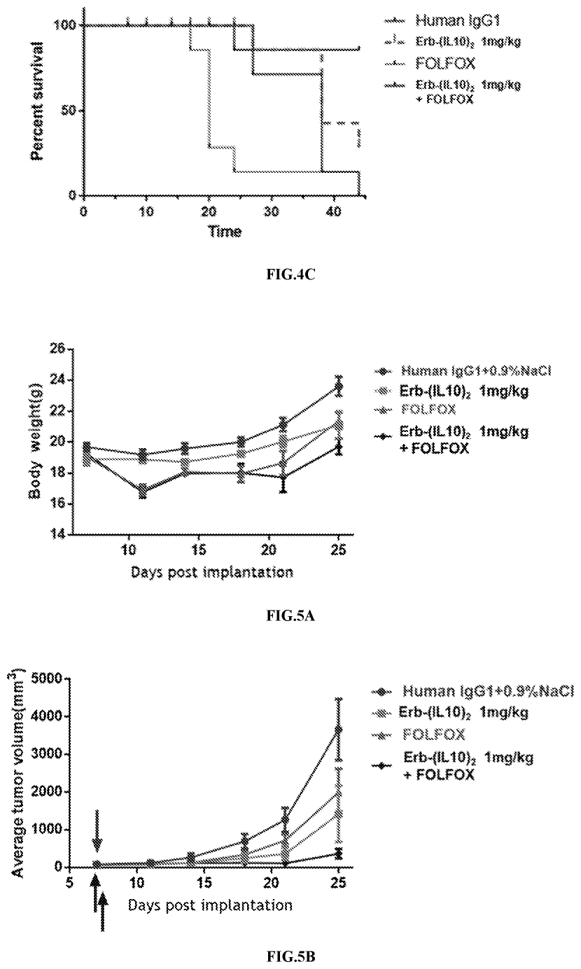

### DETAILED DESCRIPTION

Before the embodiments of the disclosure are described, it is to be understood that such embodiments are provided by way of example only, and that various alternatives to the embodiments of the disclosure described herein may be employed in practicing the disclosure. Numerous variations, changes, and substitutions will now occur to those skilled in the art without departing from the disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the disclosure.

The singular form “a,” “an” and “the,” as used herein, generally include plural references unless the context clearly dictates otherwise.

The term “immunoconjugate”, as used herein, generally refers to a proteinaceous molecule formed by the conjugation of one or more antibodies or a fragment thereof to one or more second molecules. The second molecule may be the same or different, and may include for example, effector proteins.

The term “proteinaceous,” as used herein, generally refers to a material or molecule that is of, relating to, resembling, or being a polypeptide or a protein. For example, a immunoconjugate of the present disclosure may be a heterodimer protein, or a heterodimer comprising two or more polypeptides.

The term “heterodimer,” as used herein, generally refers to a molecule (e.g. a proteinaceous molecule) composed of two different members. The two members of a heterodimer may differ in structure, function, activity and/or composition. For example, the two different members may comprise polypeptides differing in the order, number, or kind of amino acid residues forming these polypeptides. Each of the two different members of a heterodimer may independently comprise one, two or more units, polypeptide chains, or moieties.

The term “targeting moiety,” as used herein, generally refers to a molecule, complex or aggregate, that binds specifically, selectively or preferentially to a target molecule, cell, particle, tissue or aggregate. For example, a targeting moiety may be an antibody, antigen-binding antibody fragment, bispecific antibody or other antibody-based molecule or compound. Other examples of targeting moieties may include, but are not limited to, aptamers, avimers, receptor-binding ligands, nucleic acids, biotin-avidin binding pairs, binding peptides or proteins, etc. The terms “targeting moiety” and “binding moiety” are used interchangeably herein.

The term “tumor antigen,” as used herein, generally refers to an antigenic substance produced in or by tumor cells, which may have an ability to trigger an immune response in a host. For example, a tumor antigen may be a protein, a polypeptide, a peptide, or a fragment thereof, which constitutes part of a tumor cell and is capable of inducing tumor-specific cytotoxic T lymphocytes. A tumor antigen peptide may be a peptide that is generated as a result of degradation of the tumor antigen in a tumor cell and can induce or activate tumor-specific cytotoxic T lymphocytes upon being expressed on cell surface by binding to an HLA molecule. In some embodiments, the term “tumor antigen” may also refer to biomolecules (e.g., proteins, carbohydrates, glycoproteins, etc.) that are exclusively or preferentially or differentially expressed on a cancer cell and/or are found in association with a cancer cell and thereby provide targets preferential or specific to the cancer. For example, the preferential expression can be preferential expression as compared to any other cell in the organism, or preferential expression within a particular area of the organism (e.g. within a particular organ or tissue).

The term “heterodimerization,” as used herein, generally refers to the process of forming a heterodimer between two different members (e.g., two non-identical polypeptides), such as through complexation, association, or aggregation, with or without formation of covalent bonds between the two different members.

The term “covalent bond,” as used herein, generally refers to a chemical bond formed between atoms by the sharing of electrons. For example, a covalent bond may be polar or non-polar. In some embodiments, a covalent bond is a disulfide bond.

The term “non-covalent pairwise affinity,” as used herein, generally refers to that dimerization sequences or heterodimerization sequences capable of binding each other via non-covalent interaction, e.g., via ion pairs, hydrogen bonds, dipole-dipole interactions, charge transfer interactions, 7C-7C interactions, cation-n-electron interactions, van der Waals interactions and disperse interactions, hydrophobic (lipophilic) interactions, complex formation (e.g., complex formation of transition metal cations), or a combination of these interactions.

The term “linker,” as used herein, generally refers to a synthetic amino acid sequence that connects or links two polypeptide sequences, e.g., that link two polypeptide domains. A linker may connect two amino acid sequences via peptide bonds. In some embodiments, a linker of the present disclosure connects a biologically active moiety to a second moiety in a linear sequence.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component. The terms may apply to amino acid polymers in which one or more amino acid residue is an artificial chemical analogue of a corresponding naturally occurring amino acid, as well as to naturally occurring amino acid polymers. The terms may also include variants on the traditional peptide linkage joining the amino acids making up the polypeptide. For example, the “peptides,” “polypeptides,” and “proteins” may be chains of amino acids whose alpha carbons are linked through peptide bonds. The terminal amino acid at one end of the chain (amino terminal) therefore may have a free amino group, while the terminal amino acid at the other end of the chain (carboxy terminal) may have a free carboxyl group. As used herein, the term “amino terminus” (abbreviated N-terminus) generally refers to the free α-amino group on an amino acid at the amino terminal of a peptide or to the α-amino group (amino group when participating in a peptide bond) of an amino acid at any other location within the peptide. Similarly, the term “carboxy terminus” generally refers to the free carboxyl group on the carboxy terminus of a peptide or the carboxyl group of an amino acid at any other location within the peptide. Peptides may also include essentially any poly-amino acid including, but not limited to peptide mimetics such as amino acids joined by a ether as opposed to an amide bond.

The term “amino acid,” as used herein, generally refers to either natural and/or unnatural or synthetic amino acids, including but not limited to, the D or L optical isomers or both, amino acid analogs and peptidomimetics. Standard single or three letter codes are used to designate amino acids.

The term “variant,” when used in the context of a proteinaceous molecule (e.g., a polypeptide or a protein), generally refers to a proteinaceous molecule with sequence homology to the native biologically active protein that retains at least a portion of the therapeutic and/or biological activity of the biologically active protein. For example, a variant protein may share at least 60%, 65%, 70%, 75%, 80%, 85%, 90%, 95%, 96%, 97%, 98% or 99% amino acid sequence identity compared with the reference biologically active protein. In some embodiments, the “variant” may include proteins modified deliberately, as for example, by site directed mutagenesis, synthesis of the encoding gene, insertions, or accidentally through mutations.

The terms “conjugated,” “linked,” “fused,” and “fusion” are used interchangeably herein, and generally refer to the joining together of two or more chemical elements, sequences or components, e.g., by means including chemical conjugation or recombinant means. For example, a promoter or enhancer is operably linked to a coding sequence if it effects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting “fusion polypeptide” is a single protein containing two or more fragments that correspond to polypeptides encoded by the original ORFs (which segments are not normally so joined in nature). The “fusion site” refers to the sequence where the two or more fragments are joined together. In some cases, the fusion site can be a sequence that is identical to sequences in the two or more fragments being joined. In some cases, the fusion site can further comprise a gap segment that is not identical to either of the sequences of the two or more fragments being joined.

In the context of polypeptides, a “linear sequence” or a “sequence” is an order of amino acids in a polypeptide in an amino to carboxyl terminus direction in which residues next to each other in the sequence are contiguous in the primary structure of the polypeptide. A “partial sequence” is a linear sequence forming part of a polypeptide that is known to comprise additional residues in one or both directions.

The terms “polynucleotides,” “nucleic acids,” “nucleotides” and “oligonucleotides” are used interchangeably herein, and they generally refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “gene” and “gene fragment” are used interchangeably herein and generally refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The term “antibody,” as used herein, generally refers to a protein comprising one or more polypeptides substantially encoded by immunoglobulin genes or fragments of immunoglobulin genes. The immunoglobulin genes may include the kappa, lambda, alpha, gamma, delta, epsilon and mu constant region genes, as well as myriad immunoglobulin variable region genes. As used herein, light chains may be classified as either kappa or lambda. Heavy chains may be classified as gamma, mu, alpha, delta, or epsilon, which in turn define the immunoglobulin classes, IgG, IgM, IgA, IgD and IgE, respectively. An antibody as used in the present disclosure may have a structural unit comprising a tetramer. Each tetramer may be composed of two identical pairs of polypeptide chains, each pair having one “light” (about 25 KD) and one “heavy” chain (about 50-70 KD). The N-terminus of each chain may define a variable region of about 100 to 110 or more amino acids primarily responsible for antigen recognition. The terms “light chain variable region” (VL) and “heavy chain variable region” (VH), as used herein, generally refer to these regions of the light and heavy chains respectively. Antibodies may exist as intact immunoglobulins or as a number of well characterized fragments produced by digestion with various peptidases or expressed de novo. Thus, for example, pepsin may digest an antibody below the disulfide linkages in the hinge region to produce F(ab)′2 (a dimer of Fab which itself is a light chain joined to VH—CH1 by a disulfide bond). The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of ordinary skill in the art will appreciate that such Fab′ fragments may be synthesized de novo either chemically or by utilizing recombinant DNA methodology. Thus, the term antibody, as used herein, may also include antibody fragments either produced by the modification of whole antibodies or synthesized de novo using recombinant DNA methodologies, including, but are not limited to, Fab′2, IgG, IgM, IgA, IgE, scFv, dAb, nanobodies, unibodies, and diabodies. In some embodiments, the antibodies include, but are not limited to Fab′2, IgG, IgM, IgA, IgE, and single chain antibodies, for example, single chain Fv (scFv) antibodies in which a variable heavy and a variable light chain are joined together (directly or through a peptide linker) to form a continuous polypeptide.

The term “antigen-binding site” or “binding portion,” as used herein, generally refers to a part of an antibody that participates in antigen binding. An antigen binding site may be formed by amino acid residues of the N-terminal variable (“V”) regions of a heavy (“H”) chain and/or a light (“L”) chain. Three highly divergent stretches within the V regions of the heavy and light chains are referred to as “hypervariable regions” which are interposed between more conserved flanking stretches known as “framework regions” or “FRs”. Thus, the term “FR,” as used herein, generally refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three-dimensional space to form an antigen binding “surface”. This surface may mediate recognition and binding of the target antigen. The three hypervariable regions of each of the heavy and light chains are referred to as “complementarity determining regions” or “CDRs” and are characterized, for example by Kabat et al. Sequences of proteins of immunological interest, 4^{th} ed. U.S. Dept. Health and Human Services, Public Health Services, Bethesda, Md. (1987).

The term “homology,” “homologous” or “sequence identity,” as used herein, generally refers to sequence similarity or interchangeability between two or more polynucleotide sequences or between two or more polypeptide sequences. When using a program (e.g. Emboss Needle or BestFit) to determine sequence identity, similarity or homology between two different amino acid sequences, the default settings may be used, or an appropriate scoring matrix, such as blosum45 or blosum80, may be selected to optimize identity, similarity or homology scores. In some embodiments, polynucleotides that are homologous are those which hybridize under stringent conditions and have at least 60%, at least 65%, at least 70%, at least 80%, at least 90%, at least 95%, at least 97%, at least 98%, at least 99%, and even 100% sequence identity compared to those sequences. Polypeptides that are homologous have sequence identities of at least 80%, or at least 90%, or at least 95%, or at least 97%, or at least 98%, or have at least 99% sequence identity when sequences of comparable length are optimally aligned.

The term “effective amount” or “therapeutically effective amount” refers to an amount of a composition that is sufficient to effect the intended application, including but not limited to disease treatment. The therapeutically effective amount may vary depending upon the intended application (e.g., in vitro or in vivo), or the subject and disease condition being treated, e.g., the weight and age of the subject, the severity of the disease condition, the manner of administration and the like, which can readily be determined by one of ordinary skill in the art. The term may also apply to a dose that will induce a particular response in target cells, e.g. target gene induction, proliferation, and/or apoptosis. The specific dose will vary depending on the particular compounds chosen, the dosing regimen to be followed, whether it is administered in combination with other compounds, timing of administration, the tissue to which it is administered, and the physical delivery system in which it is carried.

The terms “treatment” or “treating,” or “palliating” or “ameliorating” is used interchangeably herein, and refer to an approach for obtaining beneficial or desired results including but not limited to a therapeutic benefit and/or a prophylactic benefit. As used herein, therapeutic benefit generally refers to eradication or reduced severity of the underlying disorder being treated. Also, a therapeutic benefit is achieved with the eradication, reduced severity or reduced incidence of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

The term “therapeutic effect,” as used herein, generally encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” “use in combination with,” and their grammatical equivalents, as used herein, generally encompass administration of two or more agents or therapies to a subject so that both agents and/or their metabolites, or both therapies are present and/or function in the subject. Co-administration includes simultaneous administration in separate compositions or forms, administration at different time pointes in separate compositions or forms, or administration in a composition in which both agents are present.

The term “agent” as used herein, generally refers to a biological, pharmaceutical, or chemical compound or other moieties. Non-limiting examples include a simple or complex organic or inorganic molecule, a peptide, a protein, an oligonucleotide, an antibody, an antibody derivative, antibody fragment, a vitamin derivative, a carbohydrate, a toxin, a chemotherapeutic compound, or an agent capable of generating/emitting radiation.

The term “interleukin,” as used herein, generally refers to a secreted protein or a signaling molecule capable of promoting the development and differentiation of T and/or B lymphocytes and/or hematopoietic cells. An interleukin may be synthesized by helper CD4 T lymphocytes, as well as through monocytes, macrophages, and endothelial cells. As used herein, an interleukin (IL) may include IL10. As used herein, the term “interleukin” may include full length interleukins, or a fragment (e.g., a truncated form) or variant thereof substantially maintaining the biological activities of a corresponding wild-type interleukin (e.g., having a biological activity that is at least 80%, at least 90%, at least 95%, at least 98%, at least 99%, or even at least 100% of the biological activity of a corresponding wild-type interleukin). An interleukin, as used herein, may be from any mammalian species. In some embodiments, the interleukin is from a species selected from the group consisting of human, horse, cattle, murine, pig, rabbit, cat, dog, rat, goat, sheep, and non-human primate. In some embodiments, the interleukin can be in a mutated form, for example, with increased or decreased affinity to its receptors.

The term “subject,” as used herein, generally refers to a human or non-human animal, including, but not limited to, a cat, dog, horse, pig, cow, sheep, goat, rabbit, mouse, rat, or monkey.

The term “EGFR family member,” as used herein, generally refers to a member of the epidermal growth factor receptor family. For example, it may be an ErbB-1 (also named as epidermal growth factor receptor (EGFR)).

The term “member” as used herein, generally refers to a polypeptide, subunit, or moiety which is one component of the immunoconjugate.

The term “Fc domain”, as used herein, generally refers to an Fc part or Fc fragment of an antibody heavy chain. For example, it may refer to the carboxyl terminal portion of an immunoglobulin heavy chain constant region, or an analog or portion thereof capable of binding an Fc receptor. As is known, each immunoglobulin heavy chain constant region comprises four or five domains. The domains are named sequentially as follows: CH1-hinge-CH2-CH3(-CH4). CH4 is present in IgM, which has no hinge region. The immunoglobulin heavy chain constant region useful in the present disclosure may comprise an immunoglobulin hinge region, and may also include a CH3 domain. For example, the immunoglobulin heavy chain constant region may comprise an immunoglobulin hinge region, a CH2 domain and a CH3 domain. In some embodiments, the Fc domain according to the present disclosure consists of the hinge-CH2-CH3 domain.

The term “Fc subunit”, as used herein, generally refers to a component of an Fc domain. For example, an Fc domain may be formed by two or more members, and each member may be considered as one Fc subunit.

The term “complexed with” as used herein, generally refers to the association (e.g., binding) of one member/subunit with another member/subunit of a molecule (e.g., an antibody). For example, a light chain may be complexed with a heavy chain to form a targeting moiety.

The term “binding specificity” as used herein, generally refers to the ability to specifically bind (e.g., immune-react with) a given target (while not binding or substantially not binding a non-target). A targeting moiety of the present disclosure may be monospecific and contain one or more binding sites which specifically bind a target or may be multispecific (e.g., bispecific or trispecific) and contain two or more binding sites which specifically bind the same or different targets.

The term “associates with” or “associated with” as used herein, generally refers to that one entity is in physical association or contact with another. For example, a first member of the immunoconjugate may “associate with” a second member covalently or non-covalently. In some embodiments, a first member of the immunoconjugate associates with a second member via an interface, and the interface is formed by amino acid residues (i.e., interface residues) from the first member and the second member, respectively.

The term “modification” as used herein, generally refers to any manipulation of the peptide backbone (e.g. amino acid sequence) or any post-translational modifications (e.g. glycosylation) of a polypeptide. For example, a modification is in comparison to the sequence of a corresponding wildtype polypeptide. A modification may be a substitution, an addition, and/or a deletion of one or more amino acids (e.g., 1, 2, 3, 4, 5, 6, 7, 8, 9, 10, or more).

The term “fusion protein” as used herein, generally refers to a polypeptide that comprises, or alternatively consists of, an amino acid sequence of a polypeptide fused directly or indirectly (e.g., via a linker) to an amino acid sequence of a heterologous polypeptide (i.e., a polypeptide unrelated to the former polypeptide or the domain thereof).

The term “C-terminus” as used herein, generally refers to the carboxy terminus of a polypeptide.

The term “N-terminus” as used herein, generally refers to the amino terminus of a polypeptide.

The term “immunoglobulin” as used herein, generally refers to a protein consisting of one or more polypeptides substantially encoded by immunoglobulin genes. The recognized immunoglobulin genes include the κ, λ, α,γ (IgG1, IgG2, IgG3, IgG4), δ, ε and μ constant region genes, as well as the myriad immunoglobulin variable region genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to antibodies, immunoglobulins may exist in a variety of other forms including, for example, Fv, Fab, Fab′ and (Fab′)2.

The term “fused in frame” as used herein, generally refers to the joining of two or more open reading frames (ORFs) to form a continuous longer ORF, in a manner that maintains the correct reading frame of the original ORFs.

The term “amino acid substitution” as used herein, generally refers to that one amino acid at a specific position of a polypeptide is replaced by another amino acid.

The term “EU index of the KABAT number” as used herein, generally refers to the index of the EU number corresponding to the amino acid sequence according to Kabat et al. (1971) Ann. NY Acad, Sci. 190:382-391 and Kabat, E. A., et al. (1991) Sequences of Proteins of Immunological Interest, Fifth Edition, U.S. Department of Health and Human Services, NIH Publication No. 91-3242.

The terms “isolated polynucleotide” and “isolated nucleic acid” are used interchangeably here, and generally refers to a polymeric form of nucleotides of any length, either deoxyribonucleotides or ribonucleotides, or analogs thereof, isolated from its native environment, or that is artificially synthesized.

The term “pharmaceutically acceptable excipient” as used herein, generally refers to any and all solvents, dispersion media, coatings, isotonic and absorption delaying agents, etc., that are compatible with pharmaceutical administration.

The terms “immunogenic cell death” and “immunogenic apoptosis” are used interchangeably herein, and generally refers to a form of cell death that induces an effective anti-tumor immune response through an activation of e.g., dendritic cells (DCs) and consequent activation of specific T cell responses. Immunogenic cell death may be characterized by secretion of damage-associated molecular patterns (DAMPs). The DAMPs may include Calreticulin (CRT), heat-shock proteins (HSPs), secreted amphoterin (HMGB1), ATP, etc. CRTs is normally in the lumen of endoplasmic reticulum (ER), and may be translocated after the induction of immunogenic apoptosis to the surface of dying cell where it functions as an “eat me” signal for professional phagocytes. HSPs comprise HSP70 and HSP90, which under stress condition may also be translocated to the plasma membrane. EIMGB1 is considered to be late apoptotic marker and its release to the extracellular space seems to be required for the optimal release and presentation of tumor antigens to dendritic cells. ATP may function as a “find-me” signal for monocytes when secreted and induces their attraction to the site of apoptosis.

The term “FOLFOX regimen” as used herein, generally refers to a chemotherapy regimen that include leucovorin calcium (calcium folinate, folinic acid, leucovorin), fluorouracil(5-FU) and oxaliplatin and which may be used in the treatment of advanced-stage and metastatic colorectal cancer. FOLFOX regimens differ in agent dosing and administration schedule and include FOLFOX 4, FOLFOX 6, modified FOLFOX 6 (mFOLFOX 6) and FOLFOX 7.

The term “fluorouracil”, “5-fluorouracil” or “5-FU” as used herein, generally refers to an antimetabolite fluoropyrimidine analog of the nucleoside pyrimidine, which used as an anti-cancer chemotherapy drug.

The term “oxaliplatin”, or “eloxatin” as used herein, generally refers to a cytostatic antineoplastic agent which is useful in the therapeutic treatment of various types of susceptible cancers and tumors.

The term “folinic acid”, “tetrahydrofolate”, “leucovorin”, “leucovorin calcium” or “calcium leucovorin” as used herein, generally refers to chemoprotectant used in combination with other chemotherapy drugs.

The term “elevated expression” as used herein, generally refers to the expression of a production which is higher in some conditions than in normal case. For example, EGFR has elevated expression in metastatic pancreatic cancer (for example, pancreatic ductal adenocarcinoma, PDAC) and metastatic colorectal cancer cells comprising with wide-type or normal cells.

### Composition Comprising a Immunoconjugate and a Chemotherapeutic Agent

In one aspect, the present disclosure provides a composition comprising an immunoconjugate and a chemotherapeutic agent. The immunoconjugate may comprise 1) one or more interleukins, and 2) an Fc domain consisting of a first Fc subunit and a second Fc subunit, and the first Fc subunit associates with the second Fc subunit to form a dimer. The one or more interleukins may be fused to the Fc domain. The chemotherapeutic agent may comprise a fluorouracil and an oxaliplatin.

For the immunoconjugate according to the present disclosure, at least one of the one or more interleukins (e.g., IL10) may be fused to an amino-terminal amino acid of the Fc domain (e.g., in frame). The immunoconjugate may comprise two or more interleukins. In some embodiments, at least two of the two or more interleukins are fused to an amino-terminal amino acid of the Fc domain. In some embodiments, one or more of the interleukins is fused to the Fc domain through a peptide linker and/or an immunoglobulin hinge region (e.g., in frame). In some embodiments, at least two of the two or more interleukins are fused to each other through a peptide linker (e.g., in frame) to form an interleukin dimer. At least one the interleukin dimer may be fused to an amino-terminal amino acid of the Fc domain. In some embodiments, the two or more interleukins are two or more copies of the same interleukin. For example, the two or more interleukins are two or more copies of IL10. Thus, in some embodiments, two IL10 are fused in frame to each other (e.g. via a peptide linker) to form an IL10 dimer, then, carboxy-terminal of the IL10 dimer may be fused (e.g., in frame, for example, via a peptide linker) to an amino-terminal amino acid of the Fc domain.

The linker may be a peptide comprising 1, 2, 3, 4, 5, 6, 7, 8, 9, 10, 11, 12, 13, 14, 15, 16, 17, 18, 19, 20, 21, 22, 23, 24, 25 or more amino acids. For example, the linker may comprise 1-10 amino acids (e.g., 1, 2, 3, 4, 5, 6, 7, 8, 9, 10 or more amino acids), 1-15 amino acids (e.g., 1-11, 12, 13, 14, 15 amino acids), 1-20 amino acids, 1-30 amino acids or more. In some embodiments, the linker comprises an amino acid sequence as set forth in SEQ ID NO: 41.

The immunoconjugate may further comprise a targeting moiety fused to the Fc domain, wherein the targeting moiety may exhibit binding specificity to a tumor antigen. The tumor antigen may be any immunogenic entity or a part thereof that is specifically expressed or present in a tumor environment or on the surface of a tumor cell. In some embodiments, the tumor antigen is selected from the group consisting of: an EGFR family member (such as EGFR).

The targeting moiety may be fused to an amino-terminal amino acid of the Fc domain. In some embodiments, the targeting moiety is fused to the Fc domain through a peptide linker or an immunoglobulin hinge region.

The targeting moiety may comprise an antigen binding domain of an antibody, for example, the antigen binding domain of an antibody may be a Fab moiety, a domain antibody or a ScFv moiety. In some embodiments, the antigen binding domain of an antibody is a Fab moiety. The antibody may be selected from the group consisting of anti-EGFR antibody.

In some embodiments, the antibody is an anti-EGFR antibody. For example, the anti-EGFR antibody may be cetuximab. In some embodiments, the targeting moiety comprises the heavy chain CDR1-3 of cetuximab, the light chain CDR1-3 of cetuximab, the heavy chain variable region of cetuximab, the light chain variable region of cetuximab, and/or the light chain of cetuximab. For example, the targeting moiety may be a Fab moiety comprising both the heavy chain variable region and the light chain variable region of cetuximab.

For example, the targeting moiety may comprise heavy chain CDRs having an amino acid sequence that is at least 60%, 70%, 75%, 80%, 85%, 90%, 95%, 96%, 97%, 98%, 99% or 100% identical to that comprised in the corresponding heavy chain CDR1-3 of cetuximab. Alternatively or additionally, the targeting moiety may comprise light chain CDRs having an amino acid sequence that is at least 60%, 70%, 75%, 80%, 85%, 90%, 95%, 96%, 97%, 98%, 99% or 100% identical to that comprised in the corresponding light chain CDR1-3 of cetuximab. For example, the targeting moiety may comprise a heavy chain variable region having an amino acid sequence that is at least 60%, 70%, 75%, 80%, 85%, 90%, 95%, 96%, 97%, 98%, 99% or 100% identical to that comprised in the corresponding heavy chain variable region of cetuximab. For example, the targeting moiety may comprise a light chain variable region having an amino acid sequence that is at least 60%, 70%, 75%, 80%, 85%, 90%, 95%, 96%, 97%, 98%, 99% or 100% identical to that comprised in the corresponding light chain variable region of cetuximab. For example, the targeting moiety may comprise a light chain having an amino acid sequence that is at least 60%, 70%, 75%, 80%, 85%, 90%, 95%, 96%, 97%, 98%, 99% or 100% identical to that comprised in the corresponding light chain of cetuximab.

The heavy chain CDR 1-3 of cetuximab are as set forth in SEQ ID NO: 52 (CDR1), SEQ ID NO: 53 (CDR2), and SEQ ID NO: 54 (CDR3), respectively. The light chain CDR 1-3 of cetuximab are as set forth in SEQ ID NO: 48 (CDR1), SEQ ID NO: 49 (CDR2), and SEQ ID NO: 50 (CDR3), respectively. The heavy chain variable region of cetuximab is as set forth in SEQ ID NO: 55. The light chain variable region of cetuximab is as set forth in SEQ ID NO: 51.

For the immunoconjugate according to the present disclosure, the Fc domain may be an IgG Fc domain. The IgG may be selected from the group consisting of IgG1, IgG2, IgG3 and IgG4. In some embodiments, the IgG is a human IgG1, and the Fc domain is a human IgG1 Fc domain (wildtype or modified).

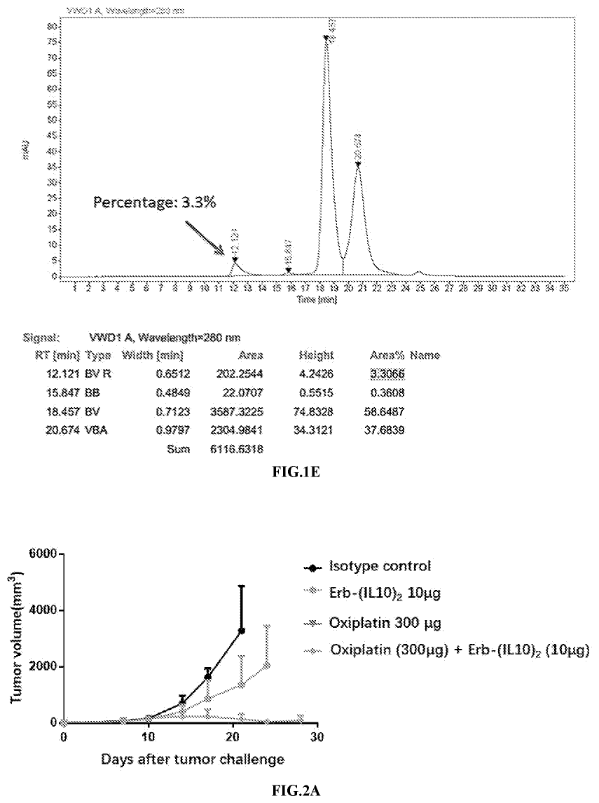

In some embodiments, the immunoconjugate is a proteinaceous homodimer consisting of two identical members. Each of the two identical members may comprise one or more interleukins (e.g., IL10) fused (e.g., in frame, such as via a peptide linker) to a subunit of the Fc domain. For example, the carboxy-terminal of the one or more interleukins may fuse to an amino-terminal amino acid of the Fc subunit. In some embodiments, the carboxy-terminal of one interleukin (e.g., IL10) is fused in frame to an amino-terminal amino acid of one of said two Fc subunits, to form one member of the homodimer, and two identical such members associate with each other via interactions between the two Fc subunits to form the homodimer (e.g., as illustrated in FIG. 3A). In some embodiments, two interleukins (e.g., two IL10) are fused in frame to each other (e.g., via a peptide linker) to form an interleukin dimer, and then, the carboxy-terminal of the interleukin dimer is fused in frame to an amino-terminal amino acid of one of said two Fc subunits, to form one member of the homodimer, and two identical such members associate with each other via interactions between the two Fc subunits to form the homodimer.

In some embodiments, the immunoconjugate is a proteinaceous heterodimer comprising a first member and a second member different from the first member, wherein the first member comprises the first Fc subunit, and the second member comprises the one or more interleukins fused to the second Fc subunit, and the first Fc subunit associates with the second Fc subunit to form the heterodimer.

In some embodiments, in the second member, at least one of the one or more interleukins is fused to the amino-terminal amino acid of the second Fc subunit.

In some embodiments, in the second member, at least two of the one or more interleukins are fused to each other to form an interleukin dimer, and the interleukin dimer is further fused to the amino-terminal amino acid of the second Fc subunit. For example, two IL10 may be fused in frame to each other (e.g., via a peptide linker, to form an IL10 dimer) and then fused in frame to the second Fc subunit, to form the second member of the proteinaceous heterodimer. For example, the carboxy-terminal of the IL10 dimer may be fused to an amino-terminal amino acid of the second Fc subunit (e.g., as illustrated in FIGS. 3B and 3C).

For example, the second member of the immunoconjugate may be a fusion protein, wherein the second Fc subunit may be fused in frame to the interleukin. In some embodiments, the carboxy-terminal of the interleukin(s) is directly or indirectly fused to an amino-terminal of the second Fc subunit to form the fusion protein. In some embodiments, the second Fc subunit is fused in frame to the interleukin(s) via a peptide linker or an immunoglobulin hinge region.

A peptide linker according to the present disclosure may be a synthetic amino acid sequence that connects or links two polypeptide sequences, e.g., via peptide bonds. In some embodiments, a linker is a peptide comprising 1, 2, 3, 4, 5, 6, 7, 8, 9, 10, 11, 12, 13, 14, 15, 16, 17, 18, 19, 20, 21, 22, 23, 24, 25 or more amino acids. For example, the linker may comprise 1-10 amino acids (e.g., 1, 2, 3, 4, 5, 6, 7, 8, 9, 10 or more amino acids), 1-15 amino acids (e.g., 1-11, 12, 13, 14, 15 amino acids), 1-20 amino acids, 1-30 amino acids or more. In some embodiments, the linker comprises an amino acid sequence as set forth in SEQ ID NO: 41. In some embodiments, the linker is resistant to proteolysis or substantially resistant to proteolysis. The hinge region may comprise an amino acid sequence as set forth in SEQ ID NO: 60.

In some embodiments, the first member further comprises the targeting moiety fused to the first Fc subunit. For example, the targeting moiety may be fused to the amino-terminal amino acid of the first Fc subunit. For example, the first member may comprise a Fab moiety of an antibody, the carboxy-terminal of which (e.g., the carboxy-terminal of the heavy chain part, such as the CH1 domain or a hinge region) is fused to amino-terminal amino acid of the first Fc subunit (e.g., as illustrated in FIG. 3C).

In some embodiments, the proteinaceous heterodimer does not comprise any targeting moiety. For example, the first member may only comprise the first Fc subunit (e.g., as illustrated in FIG. 3B).

In some embodiments, the first Fc subunit is the same as the second Fc subunit (e.g., a subunit of a wildtype human IgG1 Fc domain).

In some embodiments, the first Fc subunit is different from the second Fc subunit, and the Fc domain comprises a modification promoting heterodimerization between the first Fc subunit and the second Fc subunit. For example, the first Fc subunit may comprise a first modification, and the second Fc subunit may comprise a second modification. For example, the first modification may be in a CH3 domain of the first Fc subunit, and the second modification may be in a CH3 domain of the second Fc subunit. For example, the first modification and/or the second modification is as compared to the sequence of its corresponding wildtype Fc domain, respectively

For example, the first modification may comprise an amino acid substitution at position T366, and an amino acid substitution at one or more positions selected from the group consisting of: Y349, F405, K409, D399, K360, Q347, K392 and 5354, wherein the position of the amino acid is determined according to the EU index of the KABAT number.

In some embodiments, the first modification comprises an amino acid substitution at position T366, and an amino acid substitution at one or more positions selected from the group consisting of: Y349, F405, K409, D399, K360, Q347, K392 and S354, wherein the position of the amino acid is determined according to the EU index of the KABAT number.

For example, the amino acid substitution comprised by the first modification may be selected from the group consisting of: Y349C, Y349D, D399S, F405K, K360E, K409A, K409E, Q347E, Q347R, S354D, K392D and T366W.

In some embodiments, the first modification comprises 2-5 amino acid substitutions.

In some embodiments, the first modification comprises an amino acid substitution at a group of positions selected from any of the following groups: 1) Y349 and T366; 2) Y349, T366 and F405; 3) Y349, T366 and K409; 4) Y349, T366, F405, K360 andQ347; 5) Y349, T366, F405 and Q347; 6) Y349, T366, K409, K360 and Q347; 7) Y349, T366, K409 and Q347; 8) T366, K409 and K392; 9) T366 and K409; 10) T366, K409, Y349 and 5354; 11) T366 and F405; 12) T366, F405 and D399; and 13) T366, F405, Y349 and S354; wherein the position of the amino acid is determined according to the EU index of the KABAT number.

In some embodiments, the first modification comprises a group of amino acid substitutions selected from any of the following groups: 1) Y349C and T366W; 2) Y349C, T366W and F405K; 3) Y349C, T366W and K409E; 4) Y349C, T366W and K409A; 5) Y349C, T366W, F405K, K360E and Q347E; 6) Y349C, T366W, F405K and Q347R; 7) Y349C, T366W, K409A, K360E and Q347E; 8) Y349C, T366W, K409A and Q347R; 9) T366W, K409A and K392D; 10) T366W and K409A; 11) T366W, K409A and Y349D; 12) T366W, K409A, Y349D and S354D; 13) T366W and F405K; 14) T366W, F405K and D399S; 15) T366W, F405K and Y349D; and 16) T366W, F405K, Y349D and S354D; wherein the position of the amino acid is determined according to the EU index of the KABAT number.

In some embodiments, the second modification comprises amino acid substitutions at positions T366, L368 and Y407, as well as an amino acid substitution at one or more positions selected from the group consisting of D356, D399, E357, F405, K360, K392, K409 and Q347, wherein the position of the amino acid is determined according to the EU index of the KABAT number.

In some embodiments, the amino acid substitution comprised by the second modification is selected from the group consisting of D356C, D399S, E357A, F405K, K360E, K392D, K409A, L368A, L368G, Q347E, Q347R, T366S, Y407A and Y407V.

In some embodiments, the second modification comprises an amino acid substitution at 4-6 positions.

In some embodiments, the second modification comprises an amino acid substitution at a group of positions selected from any of the following groups: 1) D356, T366, L368, Y407 and F405; 2) D356, T366, L368 and Y407; 3) D356, T366, L368, Y407 and Q347; 4) D356, T366, L368, Y407, K360 and Q347; 5) D356, T366, L368, Y407, F405 and Q347; 6) D356, T366, L368, Y407, F405, K360 and Q347; 7) T366, L368, Y407, D399 and F405; 8) T366, L368, Y407 and F405; 9) T366, L368, Y407, F405 and E357; 10) T366, L368, Y407 and K409; 11) T366, L368, Y407, K409 and K392; and 12) T366, L368, Y407, K409 and E357; wherein the position of the amino acid is determined according to the EU index of the KABAT number.

In some embodiments, the second modification comprises a group of amino acid substitutions selected from any of the following groups: 1) D356C, T366S, L368A, Y407V and F405K; 2) D356C, T366S, L368A and Y407V; 3) D356C, T366S, L368A, Y407V and Q347R; 4) D356C, T366S, L368A, Y407V, K360E and Q347E; 5) D356C, T366S, L368A, Y407V, F405K and Q347R; 6) D356C, T366S, L368A, Y407V, F405K, K360E and Q347E; 7) T366S, L368A, Y407V, D399S and F405K; 8) T366S, L368G, Y407A and F405K; 9) T366S, L368A, Y407V, F405K and E357A; 10) T366S, L368A, Y407V and K409A; 11) T366S, L368A, Y407V, K409A and K392D; 12) T366S, L368G, Y407A and K409A; 13) T366S, L368A, Y407V, K409A and E357A; wherein the position of the amino acid is determined according to the EU index of the KABAT number.

In some embodiments, the first Fc subunit comprises the first modification, the second Fc subunit comprises the second modification, and the first modification and the second modification comprise an amino acid substitution at a group of positions selected from any of the following groups: 1) the first modification: Y349 and T366; and the second modification: D356, T366, L368, Y407 and F405; 2) the first modification: Y349, T366 and F405; and the second modification: D356, T366, L368 and Y407; 3) the first modification: Y349, T366 and K409; and the second modification: D356, T366, L368, Y407 and F405; 4) the first modification: Y349, T366, F405, K360 and Q347; and the second modification: D356, T366, L368, Y407 and Q347; 5) the first modification: Y349, T366, F405 and Q347; and the second modification: D356, T366, L368, Y407, K360 and Q347; 6) the first modification: Y349, T366, K409, K360 and Q347; and the second modification: D356, T366, L368, Y407, F405 and Q347; 7) the first modification: Y349, T366, K409 and Q347; and the second modification: D356, T366, L368, Y407, F405, K360 and Q347; 8) the first modification: T366, K409 and K392; and the second modification: T366, L368, Y407, D399 and F405; 9) the first modification: T366 and K409; and the second modification: T366, L368, Y407 and F405; 10) the first modification: T366, K409 and Y349; and the second modification: T366, L368, Y407, F405 and E357; 11) the first modification: T366, K409, Y349 and 5354; and the second modification: T366, L368, Y407, F405 and E357; 12) the first modification: T366 and F405; and the second modification: T366, L368, Y407 and K409; 13) the first modification: T366, F405 and D399; and the second modification: T366, L368, Y407, K409 and K392; 14) the first modification: T366, F405 and Y349; and the second modification: T366, L368, Y407, K409 and E357; 15) the first modification: T366, F405, Y349 and S354; and the second modification: T366, L368, Y407, K409 and E357; wherein the position of the amino acid is determined according to the EU index of the KABAT number.

In some embodiments, the first Fc subunit comprises the first modification, the second Fc subunit comprises the second modification, wherein the first modification and the second modification comprise a group of amino acid substitutions selected from any of the following groups: 1) the first modification: Y349C and T366W; and the second modification: D356C, T366S, L368A, Y407V and F405K; 2) the first modification: Y349C, T366W and F405K; and the second modification: D356C, T366S, L368A and Y407V; 3) the first modification: Y349C, T366W and K409E; and the second modification: D356C, T366S, L368A, Y407V and F405K; 4) the first modification: Y349C, T366W and K409A; and the second modification: D356C, T366S, L368A, Y407V and F405K; 5) the first modification: Y349C, T366W, F405K, K360E and Q347E; and the second modification: D356C, T366S, L368A, Y407V and Q347R; 6) the first modification: Y349C, T366W, F405K and Q347R; and the second modification: D356C, T366S, L368A, Y407V, K360E and Q347E; 7) the first modification: Y349C, T366W, K409A, K360E and Q347E; and the second modification: D356C, T366S, L368A, Y407V, F405K and Q347R; 8) the first modification: Y349C, T366W, K409A and Q347R; and the second modification: D356C, T366S, L368A, Y407V, F405K, K360E and Q347E; 9) the first modification: T366W, K409A and K392D; and the second modification: T366S, L368A, Y407V, D399S and F405K; 10) the first modification: T366W and K409A; and the second modification: T366S, L368G, Y407A and F405K; 11) the first modification: T366W, K409A and Y349D; and the second modification: T366S, L368A, Y407V, F405K and E357A; 12) the first modification: T366W, K409A, Y349D and S354D; and the second modification: T366S, L368A, Y407V, F405K and E357A; 13) the first modification: T366W and F405K; and the second modification: T366S, L368A, Y407V and K409A; 14) the first modification: T366W, F405K and D399S; and the second modification: T366S, L368A, Y407V, K409A and K392D; 15) the first modification: T366W and F405K; and the second modification: T366S, L368G, Y407A and K409A; 16) the first modification: T366W, F405K and Y349D; and the second modification: T366S, L368A, Y407V, K409A and E357A; 17) the first modification: T366W, F405K, Y349D and S354D; and the second modification: T366S, L368A, Y407V, K409A and E357A; wherein the position of the amino acid is determined according to the EU index of the KABAT number.

In some embodiments, the first Fc subunit comprises the first modification, the second Fc subunit comprises the second modification, the first modification comprises the amino acid substitutions T366W and K409A, and the second modification comprises the amino acid substitutions T366S, L368G, Y407A and F405K, wherein the position of the amino acid is determined according to the EU index of the KABAT number.

For example, the amino acid sequence of the first Fc subunit may be selected from SEQ ID NO: 17. The amino acid sequence of the interleukin may be as set forth in SEQ ID NO: 56. The amino acid sequence of the second Fc subunit may be selected from SEQ ID NO: 18.

In some embodiments, the immunoconjugate of the present disclosure is the proteinaceous heterodimer, and the amino acid sequence of the second member may be selected from SEQ ID NO: 42.

In some embodiment, the immunoconjugate may comprise a first member and a second member. The first member may comprise a first polypeptide chain and a second polypeptide chain. The second member may comprise a third polypeptide chain. The first polypeptide chain may comprise a light chain variable region of Cetuximab. The second polypeptide chain may comprise a first Fc subunit and a heavy chain variable region of Cetuximab. The third polypeptides chain may comprise a second Fc subunit and one or more interleukins. The target moiety may comprise the heavy chain variable region of Cetuximab and the light chain variable region of Cetuximab.

In some embodiment, the immunoconjugate may comprise a first member comprises a first polypeptide chain, and said second member comprises a second polypeptide chain, said first polypeptide chain may comprise an amino acid sequence as set forth in SEQ ID NO: 17, and said second polypeptide chain may comprise an amino acid sequence as set forth in SEQ ID NO: 42.

Different components of the composition may be independently packaged (e.g., not mixed from each other before administration) or pre-mixed and packaged in the same packaging unit.

The composition of the present disclosure may be a pharmaceutical composition and may further comprise a pharmaceutically acceptable excipient. Examples of pharmaceutically acceptable excipients include, but are not limited to inert solid diluents and fillers, diluents, sterile aqueous solution and various organic solvents, permeation enhancers, solubilizers and adjuvants.

In some embodiments, the pharmaceutical composition is formulated for oral administration, intravenous administration, intramuscular administration, in-situ administration at the site of a tumor, inhalation, rectal administration, vaginal administration, transdermal administration, or administration via subcutaneous repository.

The compositions of the present disclosure may comprise a therapeutically effective amount of the active agent (e.g., the immunoconjugate and the chemotherapeutic agent). A therapeutically effective amount is an amount of the subject composition capable of preventing and/or curing (at least partially) a condition or disorder (e.g., cancer) and/or any complications thereof in a subject suffering from or having a risk of developing said condition or disorder. The specific amount/concentration of the active agent comprised may vary according to the method of administration and the need of a patient, and can be determined based on e.g., volume, viscosity, and/or body weight of a patient etc.

### Method and Use for Cancer Treatment

In another aspect, the present disclosure provides an immunoconjugate for the use of treating cancer in combination with a chemotherapeutic agent. The immunoconjugate may comprise 1) one or more interleukins, and 2) an Fc domain consisting of a first Fc subunit and a second Fc subunit, and the first Fc subunit associates with the second Fc subunit to form a dimer. The one or more interleukins may be fused to the Fc domain.

In another aspect, the present disclosure provides a use of an immunoconjugate in combination with a chemotherapeutic agent in the preparation of a medicament for treating cancer in a subject in need thereof. The immunoconjugate may comprise 1) one or more interleukins, and 2) an Fc domain consisting of a first Fc subunit and a second Fc subunit, and the first Fc subunit associates with the second Fc subunit to form a dimer. The one or more interleukins may be fused to the Fc domain.

In a further aspect, the present disclosure provides a method for treating cancer in a subject in need thereof. The method comprising administering to said subject (a) an effective amount of an immunoconjugate in other parts of the present disclosure, and (b) an effective amount of a chemotherapeutic agent in other parts of the present disclosure. The immunoconjugate may comprise 1) one or more interleukins, and 2) an Fc domain consisting of a first Fc subunit and a second Fc subunit, and the first Fc subunit associates with the second Fc subunit to form a dimer. The one or more interleukins may be fused to the Fc domain.

The immunoconjugate is as defined in other parts of the present disclosure. For example, the immunoconjugate may be that comprised in the composition of the present disclosure.

The immunoconjugate may be administered to the subject subsequent to the administration of the chemotherapeutic agent. In some embodiments, more than one dose of the chemotherapeutic agent is administered to the subject, and the immunoconjugate is administered to the subject subsequent to the administration of the last (or final) dose of the chemotherapeutic agent.

For example, 2-15 doses (e.g., 2-14 doses, 2-13 doses, 2-12 doses, 2-11 doses, 2-10 doses, 2-9 doses, 2-8 doses, 3-10 doses, 4-10 doses, or 4-12 doses) of the chemotherapeutic agent may be administered to the subject, and the immunoconjugate may be administered to the subject subsequent to the administration of the last (or final) dose of chemotherapeutic agent.

The immunoconjugate may be administered to the subject no more than 10 days (e.g., no more than 9 days, no more than 8 days, no more than 7 days, no more than 6 days, no more than 5 days, no more than 4 days, no more than 3 days, no more than 2 days, no more than 1 day or immediately) after the administration of the chemotherapeutic agent (for example, after administration of the last dose of the chemotherapeutic agent). In some embodiments, the immunoconjugate is administered to the subject 0-7 days (e.g., 0-6 days, 0-5 days, 0-4 days, 0-3 days, 0-2 days, or 0-1 day) after the administration of the chemotherapeutic agent.

The immunoconjugate may be administered to the subject for two or more times (e.g., at least 2 times, at least 3 times, at least 4 times or more).

The cancer may comprise a solid tumor. For example, the cancer may be selected from the group consisting of a colorectal cancer, a pancreas cancer, and a melanoma.

In some embodiments, the cancer is within the body of a subject, e.g., a cancer or cancer cell within a human or in a non-human animal (e.g., a mammal). In some embodiments, the mammal is a human. In some embodiments, the mammal is a mouse, a rat, a cat, a dog, a rabbit, a pig, a sheep, a horse, a bovine, a goat, a gerbil, a hamster, a guinea pig, a monkey or any other mammal. Many such mammals may be subjects that are known to the art as preclinical models for certain diseases or disorders, including solid tumors and/or other cancers (e.g., Talmadge et al., 2007 Am. J. Pathol. 170:793; Kerbel, 2003 Canc. Biol. Therap. 2(4 Suppl 1): S134; Man et al., 2007 Canc. Met. Rev. 26:737; Cespedes et al., 2006 Clin. TransL Oncol. 8:318).

The Present Disclosure Also Includes the Following Embodiments:

1. A composition comprising an immunoconjugate and a chemotherapeutic agent, wherein: said immunoconjugate comprises 1) one or more interleukins, and 2) an Fc domain consisting of a first Fc subunit and a second Fc subunit, said first Fc subunit associates with said second Fc subunit to form a dimer; said one or more interleukins are fused to said Fc domain; and wherein said chemotherapeutic agent comprises a fluorouracil and/or an oxaliplatin.

2. The composition according to embodiment 1, wherein at least one of said one or more interleukins is fused to an amino-terminal amino acid of said Fc domain.

3. The composition according to any one of embodiments 1-2, wherein said immunoconjugate comprises two or more interleukins.

4. The composition according to embodiment 3, wherein at least two of said two or more interleukins are fused to an amino-terminal amino acid of said Fc domain.

5. The composition according to any one of embodiments 1-4, wherein one or more of said interleukins is fused to said Fc domain through a peptide linker and/or an immunoglobulin hinge region.

6. The composition according to any one of embodiments 3-5, wherein at least two of said two or more interleukins are fused to each other through a peptide linker to form an interleukin dimer.

7. The composition according to embodiment 6, wherein at least one said interleukin dimer is fused to an amino-terminal amino acid of said Fc domain.

8. The composition according to any one of embodiments 3-7, wherein said two or more interleukins are two or more copies of the same interleukin.

9. The composition according to embodiment 8, wherein said two or more interleukins are two or more copies of IL10.

10. The composition according to embodiment 1-9, wherein said one or more interleukins comprise one or more IL10.

11. The composition according to any one of embodiments 1-10, wherein said immunoconjugate further comprises a targeting moiety fused to said Fc domain, wherein said targeting moiety exhibits binding specificity to a tumor antigen.

12. The composition according to embodiment 11, wherein said targeting moiety is fused to an amino-terminal amino acid of said Fc domain.

13. The composition according to any one of embodiments 11-12 wherein said targeting moiety is fused to said Fc domain through a peptide linker or an immunoglobulin hinge region.

14. The composition according to any one of embodiments 11-13, wherein said targeting moiety comprises an antigen binding domain of an antibody.

15. The composition according to embodiment 14, wherein said antigen binding domain of an antibody is a Fab moiety.

16. The composition according to any one of embodiments 11-15, wherein said tumor antigen is EGFR

17. The composition according to any one of embodiments 11-16, wherein said targeting moiety comprises an antigen-binding domain of an anti-EGFR antibody.

18. The composition according to embodiment 17, wherein said anti-EGFR antibody is cetuximab.

19. The composition according to embodiment 18, wherein said targeting moiety comprises the heavy chain CDR1-3 of cetuximab. HCDR1 comprises an amino acid sequence as set forth in SEQ ID NO: 52, HCDR2 comprises an amino acid sequence as set forth in SEQ ID NO: 53, and HCDR3 comprises an amino acid sequence as set forth in SEQ ID NO: 54.

20. The composition according to any one of embodiments 18-19, wherein said targeting moiety comprises the light chain CDR1-3 of cetuximab. LCDR1 comprises an amino acid sequence as set forth in SEQ ID NO: 48, LCDR2 comprises an amino acid sequence as set forth in SEQ ID NO: 49, and LCDR3 comprises an amino acid sequence as set forth in SEQ ID NO: 50.

21. The composition according to any one of embodiments 18-20, wherein said targeting moiety comprises the heavy chain variable region of cetuximab, and the heavy chain variable region comprises an amino acid sequence as set forth in SEQ ID NO: 55.

22. The composition according to any one of embodiments 18-21, wherein said targeting moiety comprises the light chain variable region of cetuximab, and the light variable region comprises an amino acid sequence as set forth in SEQ ID NO:51.

23. The composition according to any one of embodiments 1-22, wherein said Fc domain is an IgG Fc domain.

24. The composition according to embodiment 23, wherein said IgG is an IgG1.

25. The composition according to embodiment 24, wherein said IgG is a human IgG1.

26. The composition according to any one of embodiments 1-25, wherein said immunoconjugate is an asymmetric immunoconjugate comprising a first member and a second member different from said first member, wherein said first member comprises said first Fc subunit, and said second member comprises said one or more interleukins fused to said second Fc subunit, and said first Fc subunit associates with said second Fc subunit to form said dimer of the Fc domain.

27. The composition according to embodiment 26, wherein in said second member, at least one of said one or more interleukins is fused to the amino-terminal amino acid of said second Fc subunit.

28. The composition according to embodiment 26-27, wherein in said second member, at least two of said one or more interleukins are fused to each other to form an interleukin dimer, and said interleukin dimer is further fused to the amino-terminal amino acid of said second Fc subunit.

29. The composition according to any one of embodiments 26-28, wherein said first member further comprises said targeting moiety fused to said first Fc subunit.

30. The composition according to embodiment 29, wherein in said first member, said targeting moiety is fused to the amino-terminal amino acid of said first Fc subunit.

31. The composition according to any one of embodiments 1-30, wherein said first Fc subunit is different from said second Fc subunit, and said Fc domain comprises a modification promoting heterodimerization between said first Fc subunit and said second Fc subunit.

32. The composition according to embodiment 31, wherein said first Fc subunit comprises a first modification, and said second Fc subunit comprises a second modification.

33. The composition according to embodiment 32, wherein said first modification comprises an amino acid substitution at position T366, and an amino acid substitution at one or more positions selected from the group consisting of: Y349, F405, K409, D399, K360, Q347, K392 and 5354, wherein the position of the amino acid is determined according to the EU index of the KABAT number.

34. The composition according to embodiment 33, wherein the amino acid substitution comprised by the first modification is selected from the group consisting of: Y349C, Y349D, D399S, F405K, K360E, K409A, K409E, Q347E, Q347R, S354D, K392D and T366W, wherein the position of the amino acid is determined according to the EU index of the KABAT number.

35. The composition according to any one of embodiments 32-34, wherein said first modification comprises 2-5 amino acid substitutions.

36. The composition according to any one of embodiments 32-35, wherein said first modification comprises an amino acid substitution at a group of positions selected from any of the following groups: 1) Y349 and T366; 2) Y349, T366 and F405; 3) Y349, T366 and K409; 4) Y349, T366, F405, K360 and Q347; 5) Y349, T366, F405 and Q347; 6) Y349, T366, K409, K360 and Q347; 7) Y349, T366, K409 and Q347; 8) T366, K409 and K392; 9) T366 and K409; 10) T366, K409, Y349 and 5354; 11) T366 and F405; 12) T366, F405 and D399; and 13) T366, F405, Y349 and S354; wherein the position of the amino acid is determined according to the EU index of the KABAT number.

37. The composition according to any one of embodiments 32-36, wherein said first modification comprises a group of amino acid substitutions selected from any of the following groups: 1) Y349C and T366W; 2) Y349C, T366W and F405K; 3) Y349C, T366W and K409E; 4) Y349C, T366W and K409A; 5) Y349C, T366W, F405K, K360E and Q347E; 6) Y349C, T366W, F405K and Q347R; 7) Y349C, T366W, K409A, K360E and Q347E; 8) Y349C, T366W, K409A and Q347R; 9) T366W, K409A and K392D; 10) T366W and K409A; 11) T366W, K409A and Y349D; 12) T366W, K409A, Y349D and S354D; 13) T366W and F405K; 14) T366W, F405K and D399S; 15) T366W, F405K and Y349D; and 16) T366W, F405K, Y349D and S354D; wherein the position of the amino acid is determined according to the EU index of the KABAT number.

38. The composition according to any one of embodiments 32-37, wherein said second modification comprises amino acid substitutions at positions T366, L368 and Y407, as well as an amino acid substitution at one or more positions selected from the group consisting of D356, D399, E357, F405, K360, K392, K409 and Q347, wherein the position of the amino acid is determined according to the EU index of the KABAT number.

39. The composition according to embodiment 39, wherein the amino acid substitution comprised by the second modification is selected from the group consisting of D356C, D399S, E357A, F405K, K360E, K392D, K409A, L368A, L368G, Q347E, Q347R, T366S, Y407A and Y407V, wherein the position of the amino acid is determined according to the EU index of the KABAT number.

40. The composition according to any one of embodiments 32-39, wherein the second modification comprises 4-6 amino acid substitutions.

41. The composition according to any one of embodiments 32-40, wherein the second modification comprises an amino acid substitution at a group of positions selected from any of the following groups: 1) D356, T366, L368, Y407 and F405; 2) D356, T366, L368 and Y407; 3) D356, T366, L368, Y407 and Q347; 4) D356, T366, L368, Y407, K360 and Q347; 5) D356, T366, L368, Y407, F405 and Q347; 6) D356, T366, L368, Y407, F405, K360 and Q347; 7) T366, L368, Y407, D399 and F405; 8) T366, L368, Y407 and F405; 9) T366, L368, Y407, F405 and E357; 10) T366, L368, Y407 and K409; 11) T366, L368, Y407, K409 and K392; and 12) T366, L368, Y407, K409 and E357; wherein the position of the amino acid is determined according to the EU index of the KABAT number.

42. The composition according to any one of embodiments 32-41, wherein the second modification comprises a group of amino acid substitutions selected from any of the following groups: 1) D356C, T366S, L368A, Y407V and F405K; 2) D356C, T366S, L368A and Y407V; 3) D356C, T366S, L368A, Y407V and Q347R; 4) D356C, T366S, L368A, Y407V, K360E and Q347E; 5) D356C, T366S, L368A, Y407V, F405K and Q347R; 6) D356C, T366S, L368A, Y407V, F405K, K360E and Q347E; 7) T366S, L368A, Y407V, D399S and F405K; 8) T366S, L368G, Y407A and F405K; 9) T366S, L368A, Y407V, F405K and E357A; 10) T366S, L368A, Y407V and K409A; 11) T366S, L368A, Y407V, K409A and K392D; 12) T366S, L368G, Y407A and K409A; 13) T366S, L368A, Y407V, K409A and E357A; wherein the position of the amino acid is determined according to the EU index of the KABAT number.

43. The composition according to any one of embodiments 32-42, wherein the first Fc subunit comprises the first modification, the second Fc subunit comprises the second modification, and the first modification and the second modification comprise an amino acid substitution at a group of positions selected from any of the following groups: 1) the first modification: Y349 and T366; and the second modification: D356, T366, L368, Y407 and F405; 2) the first modification: Y349, T366 and F405; and the second modification: D356, T366, L368 and Y407; 3) the first modification: Y349, T366 and K409; and the second modification: D356, T366, L368, Y407 and F405; 4) the first modification: Y349, T366, F405, K360 and Q347; and the second modification: D356, T366, L368, Y407 and Q347; 5) the first modification: Y349, T366, F405 and Q347; and the second modification: D356, T366, L368, Y407, K360 and Q347; 6) the first modification: Y349, T366, K409, K360 and Q347; and the second modification: D356, T366, L368, Y407, F405 and Q347; 7) the first modification: Y349, T366, K409 and Q347; and the second modification: D356, T366, L368, Y407, F405, K360 and Q347; 8) the first modification: T366, K409 and K392; and the second modification: T366, L368, Y407, D399 and F405; 9) the first modification: T366 and K409; and the second modification: T366, L368, Y407 and F405; 10) the first modification: T366, K409 and Y349; and the second modification: T366, L368, Y407, F405 and E357; 11) the first modification: T366, K409, Y349 and 5354; and the second modification: T366, L368, Y407, F405 and E357; 12) the first modification: T366 and F405; and the second modification: T366, L368, Y407 and K409; 13) the first modification: T366, F405 and D399; and the second modification: T366, L368, Y407, K409 and K392; 14) the first modification: T366, F405 and Y349; and the second modification: T366, L368, Y407, K409 and E357; 15) the first modification: T366, F405, Y349 and S354; and the second modification: T366, L368, Y407, K409 and E357; wherein the position of the amino acid is determined according to the EU index of the KABAT number.

44. The composition according to any one of embodiments 32-43, wherein the first Fc subunit comprises the first modification, the second Fc subunit comprises the second modification, wherein the first modification and the second modification comprise a group of amino acid substitutions selected from any of the following groups: 1) the first modification: Y349C and T366W; and the second modification: D356C, T366S, L368A, Y407V and F405K; 2) the first modification: Y349C, T366W and F405K; and the second modification: D356C, T366S, L368A and Y407V; 3) the first modification: Y349C, T366W and K409E; and the second modification: D356C, T366S, L368A, Y407V and F405K; 4) the first modification: Y349C, T366W and K409A; and the second modification: D356C, T366S, L368A, Y407V and F405K; 5) the first modification: Y349C, T366W, F405K, K360E and Q347E; and the second modification: D356C, T366S, L368A, Y407V and Q347R; 6) the first modification: Y349C, T366W, F405K and Q347R; and the second modification: D356C, T366S, L368A, Y407V, K360E and Q347E; 7) the first modification: Y349C, T366W, K409A, K360E and Q347E; and the second modification: D356C, T366S, L368A, Y407V, F405K and Q347R; 8) the first modification: Y349C, T366W, K409A and Q347R; and the second modification: D356C, T366S, L368A, Y407V, F405K, K360E and Q347E; 9) the first modification: T366W, K409A and K392D; and the second modification: T366S, L368A, Y407V, D399S and F405K; 10) the first modification: T366W and K409A; and the second modification: T366S, L368G, Y407A and F405K; 11) the first modification: T366W, K409A and Y349D; and the second modification: T366S, L368A, Y407V, F405K and E357A; 12) the first modification: T366W, K409A, Y349D and S354D; and the second modification: T366S, L368A, Y407V, F405K and E357A; 13) the first modification: T366W and F405K; and the second modification: T366S, L368A, Y407V and K409A; 14) the first modification: T366W, F405K and D399S; and the second modification: T366S, L368A, Y407V, K409A and K392D; 15) the first modification: T366W and F405K; and the second modification: T366S, L368G, Y407A and K409A; 16) the first modification: T366W, F405K and Y349D; and the second modification: T366S, L368A, Y407V, K409A and E357A; 17) the first modification: T366W, F405K, Y349D and S354D; and the second modification: T366S, L368A, Y407V, K409A and E357A; wherein the position of the amino acid is determined according to the EU index of the KABAT number.

45. The composition according to embodiment 44, wherein the first Fc subunit comprises the first modification, the second Fc subunit comprises the second modification, the first modification comprises the amino acid substitutions T366W and K409A, and the second modification comprises the amino acid substitutions T366S, L368G, Y407A and F405K, wherein the position of the amino acid is determined according to the EU index of the KABAT number.

46. The composition according to any one of embodiments 26-45, wherein the first member does not comprise any interleukin.

47. The composition according to any one of embodiments 1-46, wherein said first Fc subunit comprises an amino acid sequence as set forth in SEQ ID NO: 17.

48. The composition according to any one of embodiments 1-47, wherein said second Fc subunit comprises an amino acid sequence as set forth in any one of SEQ ID NO: 18.

49. The composition according to any one of embodiments 1-48, wherein said interleukin is a human interleukin.

50. The composition according to any one of embodiments 1-49, wherein the interleukin comprises an amino acid sequence as set forth in any one of SEQ ID NO: 56.

51. The composition according to any one of embodiments 1-50, wherein the immunoconjugate comprises a first polypeptide chain, a second polypeptide chain and a third polypeptide chain, the first polypeptide chain comprises an amino acid sequence as set forth in SEQ ID NO: 37, the second polypeptide chain comprises an amino acid sequence as set forth in SEQ ID NO: 39, and the third polypeptide chain comprises an amino acid sequence as set forth in SEQ ID NO: 42.

52. The composition according to any one of embodiments 26-50, wherein the first member comprises a first polypeptide chain and a second polypeptide chain, the second member comprises a third polypeptide chain, the first polypeptide chain comprises an amino acid sequence as set forth in SEQ ID NO: 37, the second polypeptide chain comprises an amino acid sequence as set forth in any one of SEQ ID NO: 39, and the third polypeptide chain comprises an amino acid sequence as set forth in any one of SEQ ID NO: 42.

53. The composition according to any one of claims 26-50, wherein said first member comprises a first polypeptide chain, and said second member comprises a second polypeptide chain, said first polypeptide chain comprises an amino acid sequence as set forth in SEQ ID NO: 17, and said second polypeptide chain comprises an amino acid sequence as set forth in SEQ ID NO: 42.

54. The composition according to any one of embodiments 1-53, wherein said fluorouracil comprises 5-Fu.

55. The composition according to any one of embodiments 1-54, wherein said chemotherapeutic agent further comprises a folinic acid.

56. The composition according to any one of embodiments 1-55, wherein said chemotherapeutic agent comprises a tetrahydrofolate and/or a calcium leucovorin.

57. The composition according to any one of embodiments 1-56, wherein said chemotherapeutic agent comprises a FOLFOX regimen.

58. The composition according to any one of embodiments 1-57, wherein said fluorouracil and said oxaliplatin are not mixed with each other in the composition.

59. An immunoconjugate for the use of treating cancer in combination with a chemotherapeutic agent, wherein the immunoconjugate is as defined in any one of embodiments 1-57, and said chemotherapeutic agent is as defined in any one of embodiments 1-58.

60. The immunoconjugate according to embodiment 59, wherein said cancer is selected from pancreatic cancer and colorectal cancer.

61. The immunoconjugate according to embodiment 59, wherein said pancreatic cancer is metastatic pancreatic cancer.

62. The immunoconjugate according to embodiment 59, wherein said colorectal cancer is metastatic colorectal cancer.

63. The immunoconjugate according to any one of embodiments 59-62, wherein said cancer or a cell thereof has elevated expression of EGFR.

64. Use of an immunoconjugate in combination with a chemotherapeutic agent in the preparation of a medicament for treating cancer in a subject in need thereof, wherein said immunoconjugate is as defined in any one of embodiments 1-58, and said chemotherapeutic agent is as defined in any one of embodiments 1-58.

65. The use according to embodiment 64, wherein said cancer is selected from pancreatic cancer and colorectal cancer.

66. The use according to embodiment 65, wherein said pancreatic cancer is metastatic pancreatic cancer.

67. The use according to embodiment 65, wherein said colorectal cancer is metastatic colorectal cancer.

68. The use according to any one of embodiments 64-67, wherein said cancer or a cell thereof has elevated expression of EGFR

69. A method for treating cancer in a subject in need thereof, comprising administering to said subject (a) an effective amount of an immunoconjugate as defined in any one of embodiments 1-58, and (b) an effective amount of a chemotherapeutic agent as defined in any one of embodiments 1-57.

70. The method according to embodiment 69, wherein said immunoconjugate is administered to said subject subsequent to administration of said chemotherapeutic agent.

71. The method according to embodiment 70, wherein said immunoconjugate is administered to said subject no more than 10 days after administration of said chemotherapeutic agent.

72. The method according to any one of embodiments 70-71, wherein said immunoconjugate is administered to said subject no more than 3 days after administration of said chemotherapeutic agent.

73. The method according to any one of embodiments 69-72, wherein said immunoconjugate is administered to said subject for two or more times.

74. The method according to any one of embodiments 69-73, wherein said cancer is selected from pancreatic cancer and colorectal cancer.

75. The method according to embodiment 74, wherein said pancreatic cancer is metastatic pancreatic cancer.

76. The method according to embodiment 74, wherein said colorectal cancer is metastatic colorectal cancer

77. The method according to any one of embodiments 69-76, wherein said cancer or a cell thereof has elevated expression of EGFR.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Modification and Preparation of Nucleic Acids

### 1.1 Fc Modifications

Amino acid modifications (e.g., amino acid substitutions) were made to the interface residues of human IgG1 Fc domain to obtain the following groups of modifications (as shown in table 1 below), chain A is also referred to as Fc9 or the first Fc subunit, and chain B is also referred to as Fc6 or the second Fc subunit in the present disclosure:

TABLE 1
Groups of amino acid modifications

| Group | Fc Chain | Modifications | SEQ ID NO |
|---|---|---|---|
| 1 | A | Y349C + T366W | 1 |
| | B | D356C + T366S + L368A + Y407V + F405K | 2 |
| 2 | A | Y349C + T366W + F405K | 3 |
| | B | D356C + T366S + L368A + Y407V | 4 |
| 3 | A | Y349C + T366W + K409E | 5 |
| | B | D356C + T366S + L368A + Y407V + F405K | 2 |
| 4 | A | Y349C + T366W + K409A | 6 |
| | B | D356C + T366S + L368A + Y407V + F405K | 2 |
| 5 | A | Y349C + T366W + F405K + K360E + Q347E | 7 |
| | B | D356C + T366S + L368A + Y407V + Q347R | 8 |
| 6 | A | Y349C + T366W + F405K + Q347R | 9 |
| | B | D356C + T366S + L368A + Y407V + K360E + Q347E | 10 |
| 7 | A | Y349C + T366W + K409A + K360E + Q347E | 11 |
| | B | D356C + T366S + L368A + Y407V + F405K + Q347R | 12 |
| 8 | A | Y349C + T366W + K409A + Q347R | 13 |
| | B | D356C + T366S + L368A + Y407V + F405K + K360E + Q347E | 14 |
| 9 | A | T366W + K409A + K392D | 15 |
| | B | T366S + L368A + Y407V + D399S + F405K | 16 |
| 10 | A | T366W + K409A | 17 |
| | B | T366S + L368G + Y407A + F405K | 18 |
| 11 | A | T366W + K409A + Y349D | 19 |
| | B | T366S + L368A + Y407V + F405K + E357A | 20 |
| 12 | A | T366W + K409A + Y349D + S354D | 21 |
| | B | T366S + L368A + Y407V + F405K + E357A | 20 |
| 13 | A | T366W + F405K | 22 |
| | B | T366S + L368A + Y407V + K409A | 23 |
| 14 | A | T366W + F405K + D399S | 24 |
| | B | T366S + L368A + Y407V + K409A + K392D | 25 |
| 15 | A | T366W + F405K | 22 |
| | B | T366S + L368G + Y407A + K409A | 26 |
| 16 | A | T366W + F405K + Y349D | 27 |
| | B | T366S + L368A + Y407V + K409A + E357A | 28 |
| 17 | A | T366W + F405K + Y349D + S354D | 29 |
| | B | T366S + L368A + Y407V + K409A + E357A | 28 |

Subsequently, formation of heterodimer proteins comprising the groups of modifications listed in table 1 above were examined using a ScFv-Fc/Fc system, as explained in detail below.

First of all, human immunoglobulin gamma1 (IgG1) constant region amino acid sequence was obtained from the database Uniprot (P01857), to get wildtype human IgG1-Fc region amino acid sequence (SEQ ID NO: 30). The polynucleotide fragment encoding wild type human IgG1-Fc was obtained by RT-PCR from human PBMC total RNA (SEQ ID NO: 31, named as the Fc gene fragment). A polynucleotide fragment encoding a mouse kappaIII signal peptide (SEQ ID NO: 32) was added to the 5′ end of the Fc gene by overlapping PCR, and then subcloned into the vector pcDNA4 (Invitrogen, Cat V86220), to obtain a recombinant expression vector for expressing human IgG1-Fc in mammalian cells.

A nucleic acid molecule encoding a ScFv-Fc fusion protein (SEQ ID NO:33) was synthesized, wherein the ScFv refers to an anti-HER2 single chain antibody, the amino acid sequence of the ScFv-Fc fusion protein is as set forth in SEQ ID NO: 34. The ScFv-Fc gene fragment was then subcloned into the vector pcDNA4 (Invitrogen, Cat V86220), to obtain a recombinant expression vector for expressing the ScFv-Fc fusion protein in mammalian cells.

In some cases, a polypeptide encoding a variable region of a camel single domain antibody (VhH) was fused to the N terminal of the Fc gene fragment to obtain a fusion gene fragment (as set forth in SEQ ID NO: 35) encoding the fusion protein VhH-Fc (as set forth in SEQ ID NO: 36). It was then subcloned into the vector pcDNA4 (Invitrogen, Cat V86220), to obtain a recombinant expression vector for expressing the fusion protein VhH-Fc in mammalian cells.

Then, the amino acid modifications as listed in table 1 above were respectively introduced into the ScFv-Fc (groups 1-17), the VhH-Fc (groups 9-12, 14, 15 and 17), and the Fc gene fragment (groups 1-8) by overlapping PCR, wherein chain A refers to the Fc subunit in ScFv-Fc and chain B refers to the independent Fc subunit or the Fc subunit in VhH-Fc. The gene fragments with amino acid modifications were respectively subcloned into the vector pcDNA4 (Invitrogen, Cat V86220), to obtain recombinant expression vectors for expressing the modified ScFv-Fc fusion proteins, the modified Fc proteins, and the modified VhH-Fc fusion proteins in mammalian cells.

Then, suspend-cultured HEK293 cells (ATCC CRL-1573™) were transfected with the constructed expression vectors with PEI. For each group, the expression vector expressing the A chain (ScFv-Fc fusion protein) and that expressing the B chain (Fc protein or VhH-Fc fusion protein) were co-transfected at a ratio of 1:1. After culturing for 5-6 days, supernatant of the transient expression products was collected, and the expression products comprising corresponding protein heterodimers were preliminarily purified using ProteinA affinity chromatography. Each of the preliminarily purified expression products comprises the homodimer protein ScFv-Fc/ScFv-Fc, the homodimer protein Fc/Fc (or the homodimer protein VhH-FcNhH-Fc) and the heterodimer protein ScFv-Fc/Fc (or the heterodimer protein ScFv-Fc/VhH-Fc), present in various percentages, respectively. Since the molecular weight of these proteins (i.e., the homodimers and the heterodimers) are different, their corresponding percentage could be determined according to corresponding band intensities reflected on non-reduced SDS-PAGE gels. The intensities were quantified and the results are summarized in tables 2-5 below.

TABLE 2
Percentage of homodimer proteins and heterodimer
proteins in expression products

| | ScFv-Fc | ScFv-Fc/Fc | |
|---|---|---|---|
| Group | homodimer (%) | heterodimer (%) | Fc homodimer (%) |
| 1 | 24 | 58 | 18 |
| 2 | 10 | 70 | 20 |
| 3 | 25 | 57 | 18 |
| 4 | 10 | 77 | 13 |

TABLE 3
Percentage of homodimer proteins and heterodimer
proteins in expression products

| | ScFv-Fc | ScFv-Fc/Fc | Fc |
|---|---|---|---|
| Group | homodimer (%) | heterodimer (%) | homodimer (%) |
| 2 | 17 | 60 | 23 |
| 5 | 14 | 72 | 14 |
| 6 | 14 | 62 | 24 |
| 4 | 21 | 69 | 10 |
| 7 | 24 | 64 | 12 |
| 8 | 21 | 71 | 8 |

TABLE 4
Percentage of homodimer proteins and heterodimer
proteins in expression products

| | ScFv-Fc | ScFv-Fc/VhH-Fc | VhH-Fc |
|---|---|---|---|
| Group | homodimer (%) | heterodimer (%) | homodimer (%) |
| 4 | 13 | 68 | 19 |
| 9 | 7 | 80 | 13 |
| 10 | 15 | 85 | 0 |
| 11 | 14 | 83 | 3 |
| 12 | 10 | 84 | 6 |

TABLE 5
Percentage of homodimer proteins and heterodimer
proteins in expression products

| | ScFv-Fc | ScFv-Fc/VhH-Fc | VhH-Fc |
|---|---|---|---|
| Group | homodimer (%) | heterodimer (%) | homodimer (%) |
| 2 | 9 | 64 | 27 |
| 14 | 6 | 81 | 13 |
| 15 | 5 | 88 | 7 |
| 17 | 9 | 84 | 7 |

As can be seen from tables 2-5 above, all groups of modifications promoted heterodimer formation very effectively. For illustrative purposes, the modifications in group 10 (modifications in chain A: T366W+K409A; modifications in chain B: T366S+L368G+Y407A+F405K) were used in the following examples to generate the immunoconjugate or the protein mixtures of the present disclosure.

### 1.2 Preparation of Anti-EGFR (Cetuximab)

Full length amino acid sequences of the heavy chain and light chain of Cetuximab (also known as Erbitux or Erb, which is an antibody against epidermal growth factor receptor EGFR) were obtained, and corresponding DNA sequences encoding these amino acid sequences were obtained using online tool DNAworks (helixweb.nih.gov/dnaworks/). Then, nucleic acid molecules encoding the light chain of Cetuximab (Erb-LC) were synthesized. The amino acid sequence of Erb-LC is as set forth in SEQ ID NO: 37, and the corresponding polynucleotide sequence encoding it is as set forth in SEQ ID NO: 38. Then, point mutations (T366W and K409A) were introduced into the polynucleotide sequences encoding the Fc region of Cetuximab heavy chain gene, and nucleic acid molecules encoding the modified Cetuximab heavy chain were synthesized (referred to herein as erb-Fc9), the corresponding polypeptide encoding it was named as Erb-Fc9. The amino acid sequences of Erb-Fc9 is as set forth in SEQ ID NO: 39, and the polynucleotide sequence encoding it is as set forth in SEQ ID NO: 40.

### 1.3 Preparation of (IL10)₂-Fc6

First of all, sequence information of human interleukin 10 (IL10) (P22301) was obtained from the National Center for Biotechnology Information (NCBI), and the full length polynucleotide sequences encoding it were obtained. Then, amino acid sequences of human IgG1-Fc (i.e., residue 104 to residue 330 of P01857) were obtained according to the amino acid sequences of human immunoglobulin γ1 (IgG1) constant region (P01857) from the protein database Uniprot. Afterwards, point mutations (T366S, L368G, Y407A and F405K) were introduced into the IgG1-Fc fragment, and the polypeptide obtained thereby is referred to as Fc6. Then, a linker sequence “(GGGGS)₃” (SEQ ID NO: 41) and a hinge region sequence (SEQ ID NO: 60 were added to the N-terminus of the Fc6, to obtain linker-hinge-Fc6. The corresponding DNA sequence encoding it was then designed using online tool DNAworks (helixweb.nih.gov/dnaworks/). Then, a linker sequence “(GGGGS)₃” (SEQ ID NO: 41) was added between two copies of IL10, to obtain (IL10)₂. Polynucleotide sequences encoding (IL10)₂ were then added to the 5′ end of the polynucleotide sequences encoding the linker-hinge-Fc6, thereby obtaining and synthesizing a polynucleotide sequence encoding the fusion protein (IL10)₂-Fc6. The amino acid sequence of (IL10)₂-Fc6 is as set forth in SEQ ID NO: 42, and the polynucleotide sequence encoding it is as set forth in SEQ ID NO: 43.

### 1.4 Preparation of IL10-Fc

First of all, sequence information of human interleukin 10 (IL10) (P22301) was obtained from the National Center for Biotechnology Information (NCBI), and the full length polynucleotide sequences encoding it were obtained. Then, amino acid sequences of human IgG1-Fc (i.e., residue 104 to residue 330 of P01857) were obtained according to the amino acid sequences of human immunoglobulin γ1 (IgG1) constant region (P01857) from the protein database Uniprot. Then, a linker sequence “(GGGGS)₃” (SEQ ID NO: 41) and a hinge region sequence (SEQ ID NO: 60) were added to the N-terminus of IgG1-Fc, to obtain linker-hinge-Fc. The corresponding DNA sequence encoding it was then designed using online tool DNAworks (helixweb.nih.gov/dnaworks/). Polynucleotide sequences encoding IL10 were added to the 5′ end of the polynucleotide sequences encoding the linker-hinge-Fc, thereby obtaining and synthesizing a polynucleotide sequence encoding the fusion protein IL10-Fc. The amino acid sequence of IL10-Fc is as set forth in SEQ ID NO: 44, and the polynucleotide sequence encoding it is as set forth in SEQ ID NO: 45.

### 1.5 Preparation of Fc9

Amino acid sequences of human IgG1-Fc (i.e., residue 104 to residue 330 of P01857) were obtained according to the amino acid sequences of human immunoglobulin γ1 (IgG1) constant region (P01857) from the protein database Uniprot. Afterwards, point mutations (T366W and K409A) were introduced into the IgG1Fc fragment, and the polypeptide obtained thereby is referred to as Fc9. The amino acid sequence of Fc9 is as set forth in SEQ ID NO: 17, and the polynucleotide sequence encoding it is as set forth in SEQ ID NO: 46.

### Example 2 Construction of Recombinant Plasmids

The nucleic acid molecules (encoding Erb-Fc9, Fc9, Erb-LC (Cetuximab light chain), (IL10)₂-Fc6, and IL10-Fc) obtained according to Example 1 were digested with HindIII and EcoRI (Takara), and then sub-cloned into the vector pcDNA4/myc-HisA (Invitrogen, V863-20), respectively. The plasmids obtained were verified by sequencing, and the correct recombinant plasmids were named as: pcDNA4-Erb-Fc9, pcDNA4-Fc9, pcDNA4-Erb-LC, pcDNA4-(IL10)₂-Fc6, and pcDNA4-IL10-Fc, respectively.

### Example 3 Expression and Purification of the Immunoconjugates

Two days before transfection, 12×600mLsuspension domesticated HEK293 (ATCC, CRL-1573™) cells were prepared for transient transfection, the cells were seeded at a density of 0.8×10⁶ cells/ml. Two days later, three aliquots of cell suspension were centrifuged, and then resuspended in 600 mL Freestyle293 culture medium.

The recombinant expression vectors obtained from Example 2 were divided into the following groups:

Group1:pcDNA4-Erb-Fc9(200 μg)+pcDNA4-Erb-LC(200 μg)+pcDNA4-(IL10)₂-Fc6 (200 μg)

Group2:pcDNA4-Fc9(200 μg)+pcDNA4-(IL10)₂-Fc6 (200 μg)

Group3: pcDNA4-IL10-Fc(200 μg)

All proteins were made in transiently transfected 293F cells. Briefly, FreeStyle 293F cells (Invitrogen) were grown in 293F medium (Invitrogen), transfected with non-linearized plasmid DNA and 293Fectin reagent (Invitrogen) and grown in shaker flask batches in volumes 80-100 mL/flask at 37° C., 5% CO₂ for 6 days. All proteins were purified by one-step protein A chromatography. The quality of each protein was determined by SDS-PAGE and SEC-HPLC. Similarly, the expression and purification results of the other immunoconjugates of the present application were verified and confirmed with SDS-PAGE.

The immunoconjugates thus obtained are named as (from Group 1 to Group 3, respectively): Erb-(IL10)₂, Fc9-(IL10)₂, and (IL10-Fc)₂.

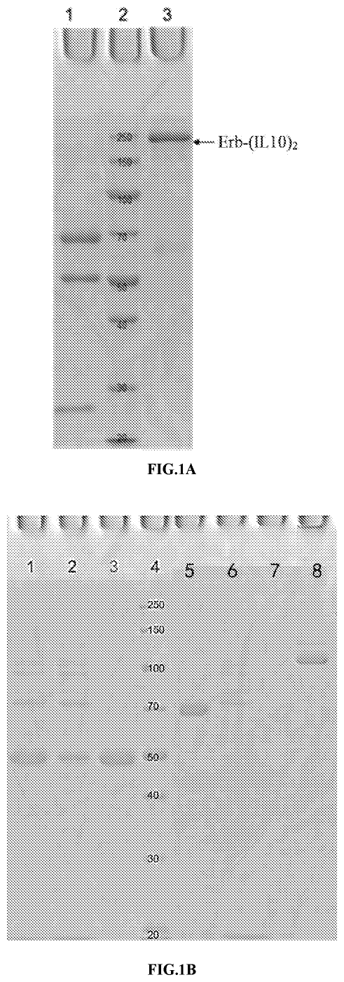

FIGS. 1A-1E show that the immunoconjugates of Erb-(IL10)₂, (IL10-Fc)₂, and Fc9-(IL10)₂ were successfully expressed and purified.

In FIG. 1A, lane 1 was loaded with Erb-(IL10)₂ (reducing); lane 2 was loaded with marker; lane 3 was loaded with Erb-(IL10)₂ (non-reducing).

In FIG. 1B, lane 1 was loaded with (IL10-Fc)₂ (original sample); lane 2 was loaded with (IL10-Fc)₂ (flow-through); lane 3 was loaded with (IL10-Fc)₂ (eluted); lane 4 was loaded with marker; lane 5 was loaded with standard positive control BSA; lane 6 was loaded with blank buffer; lane 7 was blank; and lane 8 was loaded with (IL10-Fc)₂ (eluted; non-reducing).

In FIG. 1C, lane 1 was loaded with Fc9-(IL10)₂ (original sample); lane 2 was loaded with Fc9-(IL10)₂ (flow-through); lane 3 was loaded with Fc9-(IL10)₂ (eluted); lane 4 was loaded with marker; lane 5 was loaded with standard positive control BSA; lane 6 was loaded with blank buffer; lane 7 was blank; lane 8 was loaded with Fc9-(IL10)₂ (eluted; non-reducing).

FIG. 1D shows the SEC-HPLC result, it can be seen that the percentage of undesired oligomers in the expression products of (IL10-Fc)₂ was about 27%.

FIG. 1E shows the SEC-HPLC result, it can be seen that the percentage of undesired oligomers in the expression products of Fc9-(IL10)₂ was about 3.3%.

From these results, it can be seen that the immunoconjugates of the present disclosure have been successfully produced. Interestingly, the expression products of Fc9-(IL10)₂ contain much less undesired oligomers comparing to that of (IL10-Fc)₂.

### Example 4 the Effect of Chemotherapy in Combination with the Immunoconjugate of the Present Disclosure

### 4.1 Effects of the Immunoconjugate According to the Present Disclosure in Combination with Oxiplatin

Female C57BL/6 mice were obtained from the Experimental Animal Centre of Chinese Academy of Science (Shanghai, China) at 6 to 8-week-old and maintained under specific pathogen-free conditions. All animals were used in accordance with the local ethics committee. This study was approved by the recommendations in the Guide for the Care and Use of Medical Laboratory Animals (Ministry of Health, People's Republic of China, 1998). The B16-EGFR-SIY melanoma cell line expressing human EGFR and a K^{b}-binding peptide antigen SIYRYYGL (SIY, SEQ ID NO: 47) was generated in house and grown in DMEM medium supplemented with 10% (v/v) fetal bovine serum (FBS), 100 units/ml penicillin, and 100 μg/ml streptomycin (Gibco Invitrogen).

B16-EGFR-SIY melanoma cells (5×10⁵) were inoculated subcutaneously (s.c.) into the flanks of mice and allowed to grow for about 10 days. Tumor volumes were recorded two perpendicular diameters (length and width) and calculated as V=ab²/2, where a and b are the longest and the shortest diameter, respectively. According to tumor size mice were randomly assigned into groups.

The mice bearing the tumors were divided into several groups with 5 mice per group: Group isotype control, wherein the mice were treated with 0.5 mg/kg human IgG1(10 μg/mouse); Group Erb-(IL10)₂, wherein the mice were treated with 0.5 mg/kg (10 μg/mouse) Erb-(IL10)₂; Group oxiplatin, wherein the mice were treated with 15 mg/kg (300 μg/mouse) oxiplatin; Group Erb-(IL10)₂+oxiplatin, wherein the mice were treated with 0.5 mg/kg Erb-(IL10)₂ and 15 mg/kg oxiplatin. C57BL/6 mice were inoculated s.c. with B16-EGFR-SIY cells on day 0, Erb-(IL10)₂ was injected i.p. on the day 7, 10, 14 respectively; and oxiplatin was injected through tail vein on the day 7.

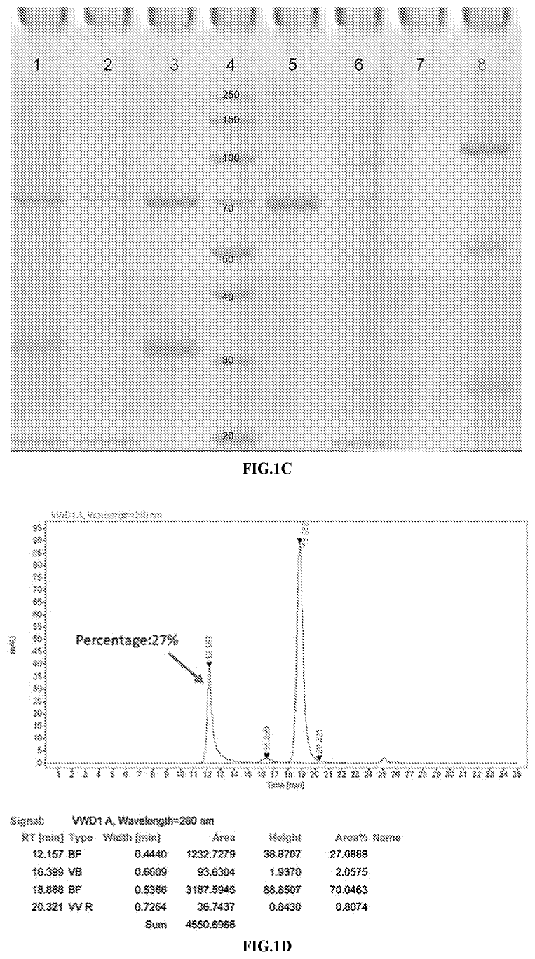

The results are shown in FIG. 2A. Due to the high toxicity of oxiplatin, all of the mice in Group oxiplatin were dead after 14 days (see FIG. 2A). It can be seen that a synergistic effect in suppressing tumor growth was observed for the combination of Erb-(IL10)₂ with oxiplatin (see FIG. 2A).

FIG. 2D shows the effects of Erb-(IL10)₂, oxiplatin, and the combination of Erb-(IL10)₂ with oxiplatin on survival rate.

Similarly, the mice bearing tumors were divided into several groups with 5 mice per group: Group Isotype control, wherein the mice were treated with 0.5 mg/kg human IgG1 (10m/mouse); Group Fc9-(IL10)₂, wherein the mice were treated with 0.5 mg/kg (10 μg/mouse) Fc9-(IL10)₂; Group oxiplatin, wherein the mice were treated with 15 mg/kg (300m/mouse) oxiplatin; Group Fc9-(IL10)₂+oxiplatin, wherein the mice were treated with 0.5 mg/kg Fc9-(IL10)₂ and 15 mg/kg (300m/mouse) oxiplatin. C57BL/6 mice were inoculated s.c. with B16-EGFR-SIY cells on day 0, Fc9-(IL10)₂ was injected i.p. on the day 7, 10, 14 respectively; and oxiplatin was injected through tail vein on the day 7.

The results are shown in FIG. 2B. Due to the high toxicity of oxiplatin, all of the mice in Group oxiplatin and Group Fc9-(IL10)₂ and oxiplatin were dead after 14 days (see FIG. 2C). Thus, the synergistic effect cannot be compared between the groups.

FIG. 2C shows the effects of Fc9-(IL10)₂, oxiplatin, and the combination of Fc9-(IL10)₂ with oxiplatin on survival rate.

### Example 5 the Effect of FOLFOX in Combination with the Immunoconjugate of the Present Disclosure

### 5.1 Effects of the Immunoconjugate According to the Present Disclosure in Combination with FOLFOX in Pancreatic Tumor Model

The C57BL/6 mice were inoculated s.c. with pancreatic tumor cell on day 0 to obtain pancreatic tumor model. The mice bearing the tumors were divided into several groups: Group isotype control, wherein the mice were treated with human IgG1; Group Erb-(IL10)₂, wherein the mice were treated Erb-(IL10)₂; Group FOLFOX, wherein the mice were treated with FOLFOX; Group Erb-(IL10)₂+FOLFOX, wherein the mice were treated with Erb-(IL10)₂ and FOLFOX. Erb-(IL10)₂ was injected i.p. and FOLFOX was injected through tail vein.

It could be seen that a synergistic effect in suppressing tumor growth was observed for the combination of Erb-(IL10)₂ with FOLFOX. It indicates that the combination of Erb-(IL10)₂ with FOLFOX has a synergistic effect in treating metastatic pancreatic cancer.

### 5.2 Effects of the Immunoconjugate According to the Present Disclosure in Combination with FOLFOX in Colorectal Tumor Model

The 70 C57BL/6 mice were inoculated s.c. with colorectal tumor MC38-EGFR-5 cells on day 0 to obtain the colorectal tumor model. The mice bearing the tumors were divided into several groups: Group isotype control, wherein the mice were treated with human IgG1; Group Erb-(IL10)₂, wherein the mice were treated Erb-(IL10)2; Group FOLFOX, wherein the mice were treated with FOLFOX; Group Erb-(IL10)2+FOLFOX, wherein the mice were treated with Erb-(IL10)₂ and FOLFOX. C57BL/6 mice were inoculated s.c. with MC38-EGFR-5 cells on day 0; and oxiplatin was injected i.v. on the day 7, Erb-(IL10)₂ was injected after 8h. Leucovorin calcium and 5-FU were injected on day 8, i.p. The dosage, route and schedule of administration were listed in table 6.

TABLE 6
Scheme of administration

| | | Dose | | Dosing | |
|---|---|---|---|---|---|
| Group | Treatment | (mg/kg) | Drug Information | Route | Schedule |
| 1 | Human IgG1 Vehicle | 1 | 12.658 mg/ml saline | i.p. | Day 8, D 11, |
| | | | | | D 15 Day 7 |
| 2 | Erb-(IL10)₂ | 1 | 10 mg/ml Erb-(IL10)₂ | i.p. | Day 8, D 11, |
| | | | | | D 15 |
| 3 | FOLFOX (leucovorin | 6, 90, 50 | FOLFOX (MCE) | i.v.&i.p. | Day 7 |
| | calcium, 5-FU and | | | | |
| | oxaliplatin) | | | | |
| 4 | Erb-(IL10)₂ + | 1 | FOLFOX (MCE) + | i.v.&i.p. | Day 8, D 11, |
| | FOLFOX | 6, 90, 50 | 10 mg/ml Erb-(IL10)₂ | | D 15 Day 7 |

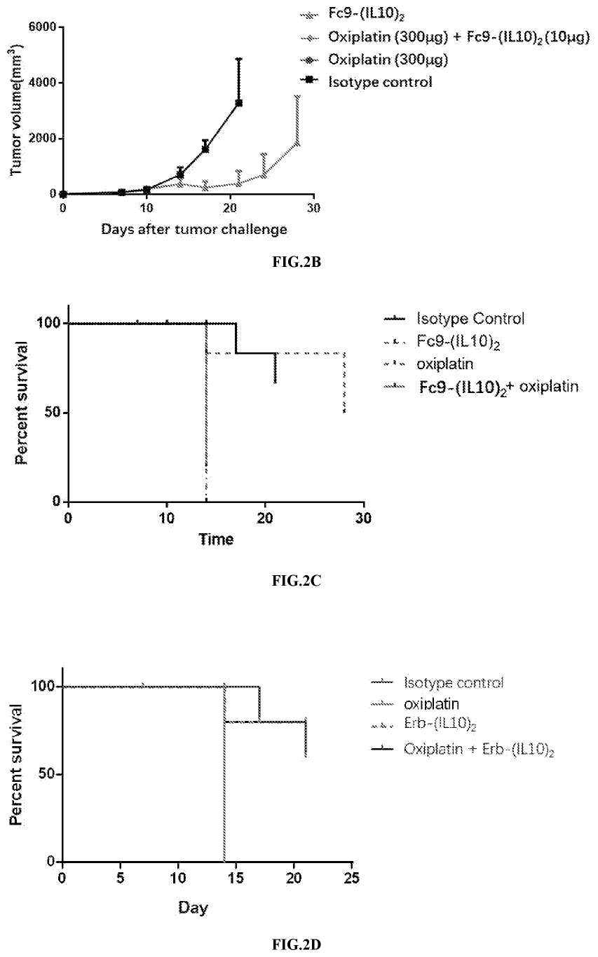

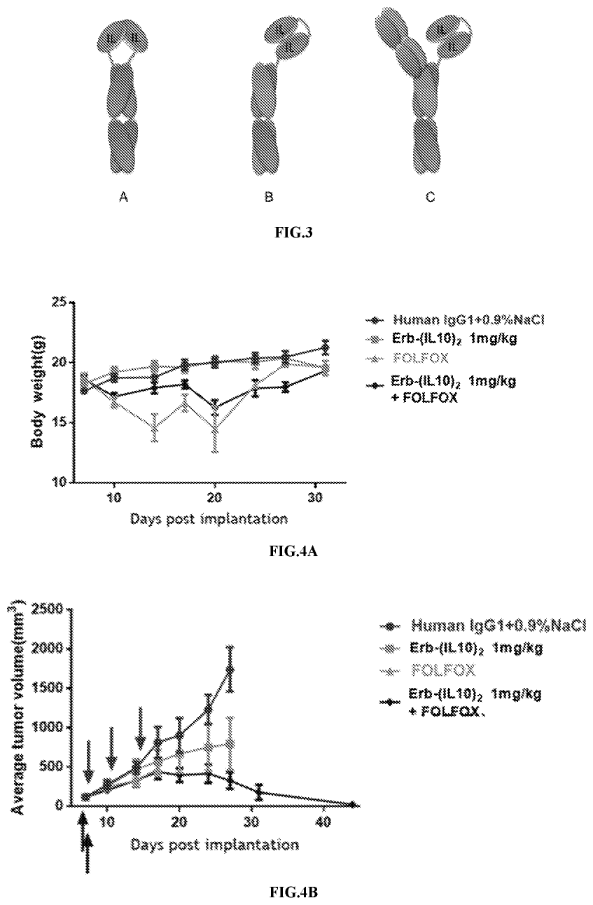

FIG. 4A and FIG. 4B shows that the combination of Erb-(IL10)₂ and FOLFOX can inhibit the tumor growth. FIG. 4C shows that the combination of Erb-(IL10)₂ and FOLFOX can prolong the survival rate.

It could be seen that a synergistic effect in suppressing tumor growth was observed for the combination of Erb-(IL10)₂ with FOLFOX. It indicates that the combination of Erb-(IL10)₂ with FOLFOX has a synergistic effect in treating metastatic colorectal cancer.

### Example 6 the Effect of Fluorouracil in Combination with the Immunoconjugate of the Present Disclosure

### 6.1 Effects of the Immunoconjugate According to the Present Disclosure in Combination with Fluorouracil in Pancreatic Tumor Model

The C57BL/6 mice were inoculated s.c. with pancreatic tumor cell on day 0 to obtain pancreatic tumor model. The mice bearing the tumors were divided into several groups: Group isotype control, wherein the mice were treated with human IgG1; Group Erb-(IL10)₂, wherein the mice were treated Erb-(IL10)₂; Group 5-FU, wherein the mice were treated with 5-FU; Group Erb-(IL10)₂₊₅-FU, wherein the mice were treated with Erb-(IL10)₂ and 5-FU. Erb-(IL10)₂ was injected i.p. and 5-FU was injected through tail vein.

It could be seen that a synergistic effect in suppressing tumor growth was observed for the combination of Erb-(IL10)₂ with 5-FU. It indicates that the combination of Erb-(IL10)₂ with fluorouracil has a synergistic effect in treating metastatic pancreatic cancer.

### 6.2 Effects of the Immunoconjugate According to the Present Disclosure in Combination with Fluorouracil in Colorectal Tumor Model

The C57BL/6 mice were inoculated s.c. with colorectal tumor MC38-EGFR cells on day 0 to obtain the colorectal tumor model. The mice bearing the tumors were divided into several groups: Group isotype control, wherein the mice were treated with human IgG1; Group Erb-(IL10)₂, wherein the mice were treated Erb-(IL10)₂; Group 5-FU, wherein the mice were treated with 5-FU; Group Erb-(IL10)₂₊₅-FU, wherein the mice were treated with Erb-(IL10)₂ and 5-FU. Erb-(IL10)₂ was injected i.p. and 5-FU was injected through tail vein.

It could be seen that a synergistic effect in suppressing tumor growth was observed for the combination of Erb-(IL10)₂ with 5-FU. It indicates that the combination of Erb-(IL10)₂ with fluorouracil has a synergistic effect in treating metastatic colorectal cancer.

### Example 7 the Effect of Oxaliplatin in Combination with the Immunoconjugate of the Present Disclosure

### 7.1 Effects of the Immunoconjugate According to the Present Disclosure in Combination with Oxaliplatin in Pancreatic Tumor Model

The C57BL/6 mice were inoculated s.c. with pancreatic tumor cell on day 0 to obtain pancreatic tumor model. The mice bearing the tumors were divided into several groups: Group isotype control, wherein the mice were treated with human IgG1; Group Erb-(IL10)₂, wherein the mice were treated Erb-(IL10)₂; Group oxaliplatin, wherein the mice were treated with oxaliplatin; Group Erb-(IL10)₂+oxaliplatin, wherein the mice were treated with Erb-(IL10)₂ and oxaliplatin. Erb-(IL10)₂ was injected i.p. and oxaliplatin was injected through tail vein.

It could be seen that a synergistic effect in suppressing tumor growth was observed for the combination of Erb-(IL10)₂ with oxaliplatin. It indicates that the combination of Erb-(IL10)₂ with oxaliplatin has a synergistic effect in treating metastatic pancreatic cancer.

### 7.2 Effects of the Immunoconjugate According to the Present Disclosure in Combination with Oxaliplatin in Colorectal Tumor Model

The C57BL/6 mice were inoculated s.c. with colorectal tumor MC38-EGFR cells on day 0 to obtain the colorectal tumor model. The mice bearing the tumors were divided into several groups: Group isotype control, wherein the mice were treated with human IgG1; Group Erb-(IL10)₂, wherein the mice were treated Erb-(IL10)₂; Group oxaliplatin, wherein the mice were treated with oxaliplatin; Group Erb-(IL10)₂+oxaliplatin, wherein the mice were treated with Erb-(IL10)₂ and oxaliplatin. Erb-(IL10)₂ was injected i.p. and oxaliplatin was injected through tail vein.

It could be seen that a synergistic effect in suppressing tumor growth was observed for the combination of Erb-(IL10)₂ with oxaliplatin. It indicates that the combination of Erb-(IL10)₂ with oxaliplatin has a synergistic effect in treating metastatic colorectal cancer.

### Example 8 Effects of the Immunoconjugate According to the Present Disclosure in Combination with FOLFOX in Melanoma Model

60 C57BL/6 mice were inoculated s.c. with melanoma B16-EGFR-5 cells (5×10⁵ cells/mouse) on day 0 to obtain the melanoma model. The mice bearing the tumors were divided into several groups: Group isotype control, wherein the mice were treated with human IgG1; Group Erb-(IL10)₂, wherein the mice were treated Erb-(IL10)₂; Group FOLFOX, wherein the mice were treated with FOLFOX; Group Erb-(IL10)₂+FOLFOX, wherein the mice were treated with Erb-(IL10)₂ and FOLFOX. The dosage, route and schedule of administration were listed in table 7. C57BL/6 mice were inoculated s.c. with B16-EGFR-5 cells on day 0; and oxiplatin was injected i.v. on the day 7, Erb-(IL10)₂ was injected after 8h. Leucovorin calcium and 5-FU were infected on day 8, i.p.

TABLE 7
Scheme of administration

| | | Dose | | Dosing | |
|---|---|---|---|---|---|
| Group | Treatment | (mg/kg) | Drug Information | Route | Schedule |
| 1 | Human IgG1 Vehicle | 0.5 | 12.658 mg/ml saline | i.p. | Day 8 |
| | | | | | Day 7 |
| 2 | Erb-(IL10)₂ | 0.5 | 10 mg/ml Erb-(IL10)₂ | i.p. | Day 8 |
| 3 | FOLFOX (leucovorin | 6, 90, 50 | FOLFOX (MCE) | i.v.&i.p. | Day 7 |
| | calcium, 5-FU and | | | | |
| | oxaliplatin) | | | | |
| 4 | Erb-(IL10)₂ + | 0.5 | FOLFOX (MCE) + | i.v.&i.p. | Day 8 |
| | FOLFOX | 6, 90, 50 | 10 mg/ml Erb-(IL10)₂ | | Day 7 |

FIG. SA and FIG. SB shows that the combination of Erb-(IL10)₂ with FOLFOX can inhibit the tumor growth.

It could be seen that a synergistic effect in suppressing tumor growth was observed for the combination of Erb-(IL10)₂ with FOLFOX. It indicates that the combination of Erb-(IL10)₂ with FOLFOX has a synergistic effect in treating melanoma.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following embodiments define the scope of the invention and that methods and structures within the scope of these embodiments and their equivalents be covered thereby.

1. A composition comprising an immunoconjugate and a chemotherapeutic agent, wherein:
said immunoconjugate comprises 1) one or more interleukins, and 2) an Fc domain consisting of a first Fc subunit and a second Fc subunit, said first Fc subunit associates with said second Fc subunit to form a dimer; said one or more interleukins are fused to said Fc domain; and
wherein said chemotherapeutic agent comprises a fluorouracil and/or an oxaliplatin.

2. (canceled)

3. The composition according to claim 1, wherein said immunoconjugate comprises two or more interleukins, wherein at least two of said two or more interleukins are fused to an amino-terminal amino acid of said Fc domain.

4-5. (canceled)

6. The composition according to claim 1, wherein said immunoconjugate comprises two or more interleukins, wherein at least two of said two or more interleukins are fused to each other through a peptide linker to form an interleukin dimer, wherein at least one of said interleukin dimer is fused to an amino-terminal amino acid of said Fc domain.

7-8. (canceled)

9. The composition according to claim 6, wherein said two or more interleukins are two or more copies of IL10.

10. (canceled)

11. The composition according to claim 1, wherein said immunoconjugate further comprises a targeting moiety fused to said Fc domain, wherein said targeting moiety exhibits binding specificity to a tumor antigen, wherein said targeting moiety is fused to an amino-terminal amino acid of said Fc domain.

12-16. (canceled)

17. The composition according to claim 11, wherein said targeting moiety comprises an antigen-binding domain of an anti-EGFR antibody.

18-20. (canceled)

21. The composition according to claim 11, wherein said targeting moiety comprises a heavy chain variable region of cetuximab, and the heavy chain variable region comprises an amino acid sequence as set forth in SEQ ID NO: 55, wherein said targeting moiety comprises a light chain variable region of cetuximab, and the light variable region comprises an amino acid sequence as set forth in SEQ ID NO: 51.

22-25. (canceled)

26. The composition according to claim 1, wherein said immunoconjugate is an asymmetric immunoconjugate comprising a first member and a second member different from said first member, wherein said first member comprises said first Fc subunit, and said second member comprises said one or more interleukins fused to said second Fc subunit, and said first Fc subunit associates with said second Fc subunit to form said dimer of the Fc domain.

27. (canceled)

28. The composition according to claim 26, wherein in said second member, at least two of said one or more interleukins are fused to each other to form an interleukin dimer, and said interleukin dimer is further fused to an amino-terminal amino acid of said second Fc subunit, said first member further comprises a targeting moiety fused to said first Fc subunit, said targeting moiety is fused to the amino-terminal amino acid of said first Fc subunit.

29-30. (canceled)

31. The composition according to claim 1, wherein said first Fc subunit is different from said second Fc subunit, and said Fc domain comprises a modification promoting heterodimerization between said first Fc subunit and said second Fc subunit, wherein said first Fc subunit comprises a first modification, and said second Fc subunit comprises a second modification.

32-43. (canceled)

44. The composition according to claim 31, wherein said first Fc subunit comprises said first modification, said second Fc subunit comprises said second modification, wherein said first modification and said second modification comprise a group of amino acid substitutions selected from any of said following groups: 1) said first modification: Y349C and T366W; and said second modification: D356C, T366S, L368A, Y407V and F405K; 2) said first modification: Y349C, T366W and F405K; and said second modification: D356C, T366S, L368A and Y407V; 3) said first modification: Y349C, T366W and K409E; and said second modification: D356C, T366S, L368A, Y407V and F405K; 4) said first modification: Y349C, T366W and K409A; and said second modification: D356C, T366S, L368A, Y407V and F405K; 5) said first modification: Y349C, T366W, F405K, K360E and Q347E; and said second modification: D356C, T366S, L368A, Y407V and Q347R; 6) said first modification: Y349C, T366W, F405K and Q347R; and said second modification: D356C, T366S, L368A, Y407V, K360E and Q347E; 7) said first modification: Y349C, T366W, K409A, K360E and Q347E; and said second modification: D356C, T366S, L368A, Y407V, F405K and Q347R; 8) said first modification: Y349C, T366W, K409A and Q347R; and said second modification: D356C, T366S, L368A, Y407V, F405K, K360E and Q347E; 9) said first modification: T366W, K409A and K392D; and said second modification: T366S, L368A, Y407V, D399S and F405K; 10) said first modification: T366W and K409A; and said second modification: T366S, L368G, Y407A and F405K; 11) said first modification: T366W, K409A and Y349D; and said second modification: T366S, L368A, Y407V, F405K and E357A; 12) said first modification: T366W, K409A, Y349D and S354D; and said second modification: T366S, L368A, Y407V, F405K and E357A; 13) said first modification: T366W and F405K; and said second modification: T366S, L368A, Y407V and K409A; 14) said first modification: T366W, F405K and D399S; and said second modification: T366S, L368A, Y407V, K409A and K392D; 15) said first modification: T366W and F405K; and said second modification: T366S, L368G, Y407A and K409A; 16) said first modification: T366W, F405K and Y349D; and said second modification: T366S, L368A, Y407V, K409A and E357A; 17) said first modification: T366W, F405K, Y349D and S354D; and said second modification: T366S, L368A, Y407V, K409A and E357A; wherein the position of the amino acid is determined according to the EU index of the KABAT number.

45-46. (canceled)

47. The composition according to claim 1, wherein said first Fc subunit comprises an amino acid sequence as set forth in any one of SEQ ID NO: 17, said second Fc subunit comprises an amino acid sequence as set forth in any one of SEQ ID NO: 18.

48-50. (canceled)

51. The composition according to claim 1, wherein said immunoconjugate comprises a first polypeptide chain, a second polypeptide chain and a third polypeptide chain, said first polypeptide chain comprises an amino acid sequence as set forth in any one of SEQ ID NO: 37, said second polypeptide chain comprises an amino acid sequence as set forth in any one of SEQ ID NO: 39, and said third polypeptide chain comprises an amino acid sequence as set forth in any one of SEQ ID NO: 42.

52. The composition according to claim 26, wherein said first member comprises a first polypeptide chain and a second polypeptide chain, said second member comprises a third polypeptide chain, said first polypeptide chain comprises an amino acid sequence as set forth in SEQ ID NO: 37, said second polypeptide chain comprises an amino acid sequence as set forth in SEQ ID NO: 39, and said third polypeptide chain comprises an amino acid sequence as set forth in SEQ ID NO: 42.

53. The composition according to claim 26, wherein said first member comprises a first polypeptide chain, and said second member comprises a second polypeptide chain, said first polypeptide chain comprises an amino acid sequence as set forth in SEQ ID NO: 17, and said second polypeptide chain comprises an amino acid sequence as set forth in SEQ ID NO: 42.

54. The composition according to claim 1, wherein said fluorouracil comprises 5-Fu.

55. The composition according to claim 1, wherein said chemotherapeutic agent further comprises a folinic acid.

56. The composition according to claim 1, wherein said chemotherapeutic agent comprises a tetrahydrofolate and/or a calcium leucovorin.

57. The composition according to claim 1, wherein said chemotherapeutic agent comprises a FOLFOX regimen.

58. (canceled)

59. A cancer treatment composition, comprising the composition of claim 1.

60-69. (canceled)

70. A method for treating cancer in a subject in need thereof, comprising administering to said subject the composition of claim 1.

71. The method according to claim 70, wherein said immunoconjugate is administered to said subject subsequent to administration of said chemotherapeutic agent.

72-74. (canceled)

75. The method according to claim 70, wherein said cancer is selected from pancreatic cancer and colorectal cancer.

76-78. (canceled)
